(19) United States
(12) Patent Application Publication
(10) Pub. No.: US 2020/0270557 A1
(43) Pub. Date: Aug. 27, 2020

(54) HUMAN IN VITRO ORTHOTOPIC AND METASTATIC MODELS OF CANCER

(71) Applicant: PRESIDENT AND FELLOWS OF HARVARD COLLEGE, Cambridge, MA (US)

(72) Inventors: Girija GOYAL, Cambridge, MA (US); Bryan HASSELL, Arlington, MA (US); Donald E. Ingber, Boston, MA (US)

(73) Assignee: PRESIDENT AND FELLOWS OF HARVARD COLLEGE, Cambridge, MA (US)

(21) Appl. No.: 16/648,050

(22) PCT Filed: Sep. 18, 2018
(86) PCT No.: PCT/US2018/051492

§ 371 Date: Mar. 17, 2020

Related U.S. Application Data
(60) Provisional application No. 62/559,958, filed on Sep. 18, 2017.

(51) Int. Cl. C12M 3/06; C12M 3/00; C12M 1/12; C12M 1/42; C12N 5/09; C12N 5/071

(52) U.S. Cl. CPC C12M 23/16; C12M 21/08; C12M 25/02; C12N 2502/14; C12N 5/0693; C12N 5/0697; C12N 5/0688; C12M 35/08

(57) ABSTRACT

Disclosed herein are devices and methods for generating orthotopic models of cancer. The devices and methods include providing a microfluidic device having a body, the body including a first microchannel separated from a second microchannel by an at least partially porous membrane, the membrane having a first side facing the first microchannel and a second side facing the second microchannel, seeding the first side of the membrane with healthy cells and cancer cells such that the cancer cells are seeded with a differentiated tissue layer, and culturing the healthy cells and the cancer cells within the microfluidic device by flowing medium through one or more of the first and second microchannels with or without endothelium in the second channel.

### CROSS-REFERENCE TO RELATED APPLICATIONS

The present application claims the benefit of, and priority to, U.S. Provisional Application No. 62/559,958, filed Sep. 18, 2017, entitled, “HUMAN IN VITRO ORTHOTOPIC AND METASTATIC MODELS OF CANCER,” the entirety of which is hereby incorporated by reference herein in its entirety.

### GOVERNMENT SUPPORT

This invention was made with Government support under Grant No. W911NF-12-2-0036 awarded by the Defense Advanced Research Projects Agency (DARPA). The Government has certain rights in the invention.

### FIELD OF THE INVENTION

The field of the invention relates to methods for making and utilizing orthotopic cancer models.

### BACKGROUND

Prior to the instant application, development of improved cancer therapeutics required better experimental models. To meet this challenge, animal researchers have moved away from conventional subcutaneous implants because they do not mimic organ-specific differences in cancer growth or responses to therapy observed in patients. Instead, human tumor xenografts are often implanted in mice at the ‘orthotopic’ organ site from which the tumors were derived. These in vivo orthotopic cancer models better mimic tumor growth and metastasis. However, using these models, it still remains extremely difficult to identify contributions of the microenvironment to tumor growth or visualize cancer cell behaviors over time, and the organ-specific microenvironment can be still not human. Thus, there has been a search for in vitro models of human cancer that provide an alternative approach.

### SUMMARY

Aspects of the present disclosure include a method of forming an orthotopic model. The method includes providing a first microfluidic device having a body. The body includes a first microchannel separated from a second microchannel by an at least partially porous membrane. The membrane has a first side facing the first microchannel and a second side facing the second microchannel. The method further includes seeding the first side of the membrane with healthy cells and cancer cells to form a tissue layer. The method further includes culturing the healthy cells and the cancer cells within the first microfluidic device by flowing fluid through one or more of the first and second microchannels. The method is controlled so that the density of the cancer cells adhered to the first side of the membrane is in a range such that the culturing of the healthy cells and the cancer cells causes the cancer cells to integrate into the tissue layer formed of healthy cells.

Aspects of the method include at least some of said cancer cells and at least some of said healthy cells corresponding to the same organ.

Further aspects of the method include, alone and combined with the other aspects, at least some of said cancer cells and at least some of said healthy cells correspond to the different organs.

Further aspects of the method include, alone and combined with the other aspects, the cancer cells being seeded prior to said healthy cells

Further aspects of the method include, alone and combined with the other aspects, the culturing of the healthy cells and the cancer cells causing the cancer cells to form tight junctions with said healthy cells.

Further aspects of the method include, alone and combined with other aspects, the healthy cells being differentiated and said cancer cells growing more slowly in the presence of said differentiated healthy cells than in the presence of undifferentiated healthy cells.

Further aspects of the method include, alone and combined with the other aspects, the cancer cells growing within, above, and/or below the tissue layer.

Further aspects of the method include, alone and combined with the other aspects, the culturing of immune cells within the tissues grown in the first and/or second microchannels.

Further aspects of the method include, alone and combined with the other aspects, the culturing including flowing a culturing medium through the second microchannel while air is present in the first microchannel.

Further aspects of the method include, alone and combined with the other aspects, the culturing including flowing a culturing medium through the first and second microchannels.

Further aspects of the method include, alone and combined with the other aspects, the healthy cells seeded in the first channel being epithelial cells, and the method further including seeding a second healthy cell population the second microchannel, the second side of the membrane, or a combination thereof with endothelial cells to.

Further aspects of the method include, alone and combined with the other aspects, a ratio of the healthy cells to the cancer cells adhered on the first side of the membrane being between about 25:1 and about 500:1.

Further aspects of the method include, alone and combined with the other aspects, the ratio of the healthy cells to the cancer cells adhered on the first side of the membrane being about 100:1

Further aspects of the method include, alone and combined with the other aspects, a density of the cancer cells adhered to the first side of the membrane being between about 100 to about 10,000 cells/cm2.

Further aspects of the method include, alone and combined with the other aspects, the density of the cancer cells adhered to the first side of the membrane being about 3200 cells/cm2.

Further aspects of the method include, alone and combined with the other aspects, the membrane being coated with at least one attachment molecule that supports adhesion of the healthy cells, the cancer cells, or a combination thereof.

Further aspects of the method include, alone and combined with the other aspects, applying a fluidic shear force across the membrane within the first microchannel, a second microchannel, or combination thereof.

Further aspects of the method include, alone and combined with the other aspects, applying a mechanical force to the healthy cells, the cancer cells, or combination thereof.

Further aspects of the method include, alone and combined with the other aspects, applying a mechanical force to the membrane in order to apply force to the healthy cells, the cancer cells, or combination thereof.

Further aspects of the method include, alone and combined with the other aspects, applying the fluidic shear force to control growth of the cancer cells by inhibiting growth as compared to absence of the fluidic shear force.

Further aspects of the method include, alone and combined with the other aspects, the fluidic shear force, the mechanical force, or combination thereof controlling growth of the cancer cells as compared to absence of the said shear force, mechanical force, or the combination thereof.

Further aspects of the method include, alone and combined with the other aspects, the fluidic shear force mimicking a shearing force of air within a lung during breathing motions.

Further aspects of the method include, alone and combined with the other aspects, the fluidic shear force mimicking a shearing force of blood flowing through a vessel.

Further aspects of the method include, alone and combined with the other aspects, the mechanical force mimicking expansion and contraction of a lung during breathing motions.

Further aspects of the method include, alone and combined with the other aspects, the mechanical force mimics the motion of at least one portion of the intestine during peristaltic motions.

Further aspects of the method include, alone and combined with the other aspects, applying one or more agents to the healthy cells, the cancer cells, or a combination thereof, and analyzing the healthy cells, the cancer cells, or a combination thereof to determine effects of the one or more agents.

Further aspects of the method include, alone and combined with the other aspects, the one or more agents being selected from the group consisting of a small molecule, a drug or drug candidate, a chemotherapeutic, a nanoparticle, a compound, a polypeptide, a polynucleotide, or a lipid, immunomodulatory, and microbes.

Further aspects of the method include, alone and combined with the other aspects, the one or more agents being one or more anti-cancer drugs, and the analyzing being on the effects of the one or more anti-cancer drugs on the cancer cells.

Further aspects of the method include, alone and combined with the other aspects, the analyzing comprises detecting the molecular level modulation of drug action.

Further aspects of the method include, alone and combined with the other aspects, the one or more anti-cancer drugs being one or more tyrosine-kinase inhibitors.

Further aspects of the method include, alone and combined with the other aspects, applying the one or more agents to the healthy cells, the cancer cells, or a combination thereof prior to, during, and/or after the application of a fluidic shear force, mechanical force, or a combination thereof and analyzing the healthy cells, the cancer cells, or a combination thereof to determine effects of the one or more agents.

Further aspects of the method include, alone and combined with the other aspects, comparing the effects of the one or more agents with or without application of the fluidic shear force, the mechanical force, or a combination thereof.

Further aspects of the method include, alone and combined with the other aspects, evaluating the migration of cancer cells between said first and second microchannels.

Further aspects of the method include, alone and combined with the other aspects, the healthy cells being primary cells, primary cells comprising more than one primary cell type, the healthy cells being mammalian primary cells, human primary cells, primary epithelial cells, primary endothelial cells, primary stromal cells, primary lung cells, lung alveolar cells, airway epithelial cells, liver hepatocyte cells, intestinal epithelial cells, and/or sinusoidal liver endothelial cells.

Further aspects of the method include, alone and combined with the other aspects, the cancer cells being primary cancer cells, such as human primary cancer cells; cancer cells from a cancer cell line, such as the cancer cell line being established from human tissue; the cancer cells being lung cancer cells, such as non-small cell lung cancer cells, including the non-small cell lung cancer cells being non-small cell lung cancer adenocarcinoma cells; and cancer cells being metastatic cancer cells.

In some embodiments, a monolayer consisting of healthy cells and cancer cells are formed in the same channel of a chip. As one example, endothelial cells, cancer cells and epithelial cells are seeded into a device on the same day in the following exemplary order: endothelial cells are seeded into the vascular channel, prior to seeding cancer cells in the apical channel, wherein the cancer cells are at low density compared to the number of healthy cells that will be subsequently seeded into the apical channel. After the cancer cells have attached, i.e. the cancer cells will not wash off the surface of the membrane, healthy cells, i.e. epithelial cells, are seeded into the apical channel. After seeded epithelial cells begin attaching to the device membrane, and before the epithelial cells become attached to the cancer cells, the apical channel is washed with enough force to remove epithelial cells from the cancer cells but not remove many from the membrane, resulting in a monolayer of cells consisting of healthy cells and cancer cells. In other words, washing the newly seeded epithelial cell layer quickly after seeding results in a monolayer of attached cells consisting of healthy cells and cancer cells. Thus, it was discovered that when epithelial cells are not washed quickly enough after seeding, that healthy cells attach to, and stick on top of cancer cells. Such healthy cells attached to the cancer cells tend to grow poorly and eventually sloth off the cell layer into the fluid part of the channel.

In a preferred embodiment, a layer of mixed cells as a monolayer, is desired, i.e. a mosaic of healthy cells and cancer cells, in part for producing replicable relative numbers of cancer cells seeded into a chip. As one example, such reproducibility was not observed in some examples when cancer cells are seeded after epithelial cells, i.e. grow on top of an epithelial cell layer, demonstrating a wide range of adherence of cancer cells to epithelial cells, thus seeded cancer cell numbers vary widely from device to device. As another example, such reproducibility was not observed in at least 10 chips intended to be identical when a mixture of healthy cells and cancer cells (mixed prior to seeding chip) were used to seed chips. Instead, the 10 chips showed a wide range of cancer cell attachment, thus a wide range of cancer cell numbers in chips at the beginning of experiments. In part, differences in buoyancy between healthy and cancer cells are contemplated to contribute to this range of cancer cells attached to healthy cells between chips.

Further aspects of the method include, alone and combined with the other aspects, the healthy cells and the cancer cells being derived from the same tissue type.

Further aspects of the method include, alone and combined with the other aspects, the healthy cells and the cancer cells being not derived from the same tissue type.

Further aspects of the method include, alone and combined with the other aspects, contacting the healthy cells, the cancer cells, or a combination thereof with at least one agent.

Further aspects of the method include, alone and combined with the other aspects, measuring a response of the healthy cells, the cancer cells, or a combination thereof to the at least one agent.

Further aspects of the method include, alone and combined with the other aspects, extracting the cancer cells from the first microfluidic device prior to measuring the response.

Further aspects of the method include, alone and combined with the other aspects, measuring products of the cancer cells or healthy cells from effluent of the first microfluidic device.

Further aspects of the method include, alone and combined with the other aspects, assessing viability of the cancer cells after the contacting.

Further aspects of the method include, alone and combined with the other aspects, the cancer cells are breast cancer cells, colorectal cancer cells, pancreatic cancer cells, kidney cancer cells, prostate cancer cells, urothelial cancer cells, oesophageal cancer cells, head and neck cancer cells, hepatocellular cancer cells, mesothelioma cells, Kaposi's sarcoma cells, ovarian cancer cells, soft tissue sarcoma cells, glioma, melanoma cells, small-cell and non-small-cell lung cancer cells, endometrial cancer cells, basal cell carcinoma cells, transitional cell carcinoma of the urothelial tract, cervical cancer cells, endometrial cancer cells, gastric cancer cells, bladder cancer cells, uterine sarcoma cells, multiple myeloma cells, soft tissue and bone sarcoma cells, cholangiocarcinoma cells, or a cancer cells disseminated therefrom.

Further aspects of the method include, alone and combined with the other aspects, imaging the cancer cells within the first microfluidic device.

Further aspects of the method include, alone and combined with the other aspects, modifying the cancer cells to express a fluorescent protein, where the fluorescent protein promotes imaging of the cancer cells.

Further aspects of the method include, alone and combined with the other aspects, monitoring growth of the cancer cells based on the imaged cancer cells.

Further aspects of the method include, alone and combined with the other aspects, further providing a second microfluidic device in fluid connection downstream of the first microfluidic device.

Further aspects of the method include, alone and combined with the other aspects, the type of healthy cells comprised in the first microfluidic device and the second microfluidic device being different.

Further aspects of the method include, alone and combined with the other aspects, the flowing medium flowing through the first microfluidic device to the second microfluidic device.

Further aspects of the method include, alone and combined with the other aspects, the cancer cells seeded in the first microfluidic device traveling to the second microfluidic device.

Further aspects of the method include, alone and combined with the other aspects, the cancer cells seeded in the first microfluidic device being integrating into the tissue layer formed of differentiated healthy cells of the second microfluidic device.

Further aspects of the method include, alone and combined with the other aspects, the cancer cells and the healthy cells seeded in the first microfluidic device being derived from the same tissue type.

Further aspects of the method include, alone and combined with the other aspects, the cancer cells and the healthy cells seeded in the first microfluidic device being derived from a different tissue type.

Further aspects of the method include, alone and combined with the other aspects, the cancer cells seeded in the first microfluidic device and the healthy cells seeded in the second microfluidic device being derived from a different tissue type.

Further aspects of the method include, alone and combined with the other aspects, the healthy cells and the cancer cells seeded in the first microfluidic device being derived from the lung; and the healthy cells seeded in the second microfluidic device being derived from the liver.

Additional aspects of the present disclosure include a method of a) providing i) cancer cells having one or more mesenchymal-like features, ii) healthy epithelial cells, and a fluidic device having a membrane. The method further includes b) co-culturing said cancer cells and said healthy epithelial cells on a first surface of the membrane under conditions such that at least a portion of said cancer cells form tight junctions with said healthy epithelial cells.

Further aspects of the method include, alone and combined with the other aspects, the cancer cells being provided within the first microchannel on the membrane at a density range of about 100 to about 10,000 cells/cm².

Further aspects of the method include, alone and combined with the other aspects, the density range controlling the growth of the cancer cells to promote cancer cell growth compared to outside the density range.

Further aspects of the method include, alone and combined with the other aspects, the cancer cells being provided within the first microchannel on the membrane at a density about 3200 cells/cm².

Further aspects of the method include, alone and combined with the other aspects, the cancer cells being provided within the first microchannel on the membrane at a ratio of the healthy cells to cancer cells of about 25:1 and about 500:1.

Further aspects of the method include, alone and combined with the other aspects, the ratio controlling the growth of the cancer cells to promote cancer cell growth compared to outside of the ratio.

Further aspects of the method include, alone and combined with the other aspects, the cancer cells being provided within the first microchannel by seeding the first microchannel with the cancer cells prior to differentiating of the healthy cells into the differentiated layer.

Further aspects of the method include, alone and combined with the other aspects, seeding the first microchannel with the cancer cells prior to or after differentiating of the healthy cells into the differentiated layer controlling the growth of the cancer cells to promote cancer cell growth.

Further aspects of the method include, alone and combined with the other aspects, the cancer cells being seeded within the first microchannel by perfusing the first microchannel with a seeding medium containing the cancer cells.

Further aspects of the method include, alone and combined with the other aspects, the seeding medium including the healthy cells and seeds the first microchannel for forming the differentiated layer.

Further aspects of the method include, alone and combined with the other aspects, the healthy cells being seeded within the first microchannel by perfusing the first microchannel with another seeding medium containing the healthy cells during or after the perfusing of the first microchannel with the seeding medium containing the cancer cells.

Further aspects of the method include, alone and combined with the other aspects, the cancer cells being provided within the first microchannel by seeding the first microchannel with the cancer cells after differentiating of the healthy cells into the differentiated layer.

Further aspects of the method include, alone and combined with the other aspects, where seeding the first microchannel with the cancer cells after differentiating of the healthy cells into the differentiated layer controls the growth of the cancer cells to inhibit cancer cell growth.

Further aspects of the method include, alone and combined with the other aspects, the method further including differentiating said healthy epithelial cells into a differentiated layer, with the cancer cells being seeded on said membrane prior to or after differentiating of the healthy cells into the differentiated layer.

Further aspects of the method include, alone and combined with the other aspects, the seeding of the cancer cells prior to or after differentiating of the healthy cells into the differentiated layer controls the growth of the cancer cells.

Further aspects of the method include, alone and combined with the other aspects, where the cancer cells are provided after differentiating of the healthy cells into the differentiated layer.

Further aspects of the method include, alone and combined with the other aspects, where the seeding of the cancer cells after differentiating of the healthy cells into the differentiated layer controls the growth of the cancer cells to inhibit cancer cell growth.

Further aspects of the method include, alone and combined with the other aspects, the method further including continuing to co-culture until said tumor cells progress to form nodules.

Further aspects of the method include, alone and combined with the other aspects, the method further including contacting the healthy cells, the cancer cells, or a combination thereof with at least one agent.

Further aspects of the method include, alone and combined with the other aspects, the agent kills at least a portion of said cancer cells.

Further aspects of the method include, alone and combined with the other aspects, the method also including contacting the co-culture with an agent that inhibits formation of said nodules.

Further aspects of the method include, alone and combined with the other aspects, said one or more mesenchymal-like features being selected from the group consisting of expression of vimentin, expression of aSMA, and expression of n-cadherin.

Further aspects of the method include, alone and combined with the other aspects, where the at least a portion of said cancer cells transmigrate said membrane.

Further aspects of the method include, alone and combined with the other aspects, where said fluidic device is a transwell.

Further aspects of the method include, alone and combined with the other aspects, where said fluidic device is a microfluidic device.

Further aspects of the method include, alone and combined with the other aspects, where at least a portion of said cancer cells in step b) undergo a mesenchymal-epithelial transition.

Yet further aspects of the present disclosure include a method of providing a microfluidic device having a body. The body includes a first microchannel and a second microchannel separated by an at least partially porous membrane. A first surface of the membrane within the first microchannel includes cancer cells integrated into a healthy cell layer. The method further includes applying one or more mechanical forces, shearing forces, or a combination thereof to the membrane, to the cancer cells, or a combination thereof to control growth of the cancer cells.

Further aspects of the method include, alone and combined with the other aspects, the mechanical forces to the membrane causing the membrane to expand and contract, and expansion and contraction of the membrane controlling the growth of the cancer cells to inhibit growth mimicking a persister cell.

Further aspects of the method include, alone and combined with the other aspects, providing a gas within the first microchannel and a liquid within the second microchannel, where passing the gas through the first microchannel applies the shearing forces to the cancer cells, and the shearing forces control the growth of the cancer cells to inhibit growth.

Further aspects of the present disclosure include a microfluidic device. The device includes a membrane, a first cell layer formed on the first side of the membrane. The first cell layer includes first healthy cells and cancer cells, in which the cancer cells are integrated into first cell layer and have tight junctions with said healthy cells.

Further aspects of the device include, alone and combined with the other aspects, the first healthy cells being epithelial cells.

Further aspects of the device include, alone and combined with the other aspects, the cancer cells being adhered to the membrane at a cell density of about 100 to about 10,000 cells/cm².

Further aspects of the device include, alone and combined with the other aspects, the cell density being about 3200 cells/cm².

Further aspects of the device include, alone and combined with the other aspects, a ratio of the first healthy cells to the cancer cells adhered on the first side of the membrane being between about 25:1 and about 500:1.

Further aspects of the device include, alone and combined with the other aspects, the ratio being about 100:1.

Further aspects of the device include, alone and combined with the other aspects, further comprising a second cell layer formed at least on some portion of the second side of the membrane, the second cell layer comprising second healthy cells.

Further aspects of the device include, alone and combined with the other aspects, the second cell layer comprising endothelial cells

Further aspects of the device include, alone and combined with the other aspects, further being adapted to permit mechanical strain.

Further aspects of the device include, alone and combined with the other aspects, at least some of said cancer cells and at least some of said healthy cells corresponding to the same organ.

Further aspects of the device include, alone and combined with the other aspects, at least some of said cancer cells and at least some of said healthy cells corresponding to the different organs.

Further aspects of the device include, alone and combined with the other aspects, the device further including a second cell layer formed at least one the second side of the membrane, the first cell layer comprising second healthy cells.

Further aspects of the device include, alone and combined with the other aspects, said fluidic device being a transwell.

Further aspects of the device include, alone and combined with the other aspects, said fluidic device being a microfluidic device. The microfluidic device can include a first microchannel and a second microchannel. The membrane separates the first microchannel from the second microchannel. The first side facing of the membrane faces the first microchannel and the second side of the membrane faces the second microchannel.

Additional aspects of the present disclosure include a method of a) providing i) cancer cells having one or more mesenchymal-like features, ii) healthy epithelial cells, and a fluidic device having a membrane. The method includes b) co-culturing said cancer cells and said healthy epithelial cells on a first surface of the membrane under conditions such that at least a portion of said cancer cells form tight junctions with said healthy epithelial cells. The method further includes c) continuing to co-culture until at least a portion of said cancer cells lose said tight junctions with said healthy epithelial cells.

Further aspects of the method include, alone and in combination with other aspects, said one or more mesenchymal-like features being selected from the group consisting of expression of vimentin, expression of aSMA, and expression of n-cadherin.

Further aspects of the method include, alone and in combination with other aspects, where, after step c), at least a portion of said cancer cells progress to form nodules.

Further aspects of the method include, alone and in combination with other aspects, where, after step c), at least a portion of said cancer cells transmigrate said membrane.

Further aspects of the method include, alone and in combination with other aspects, where said fluidic device is a transwell.

Further aspects of the method include, alone and in combination with other aspects, where said fluidic device is a microfluidic device. The microfluidic device can include first and second microchannels separated by said membrane.

Further aspects of the method include, alone and in combination with other aspects, the method further including contacting the healthy cells, the cancer cells, or a combination thereof with at least one agent.

Further aspects of the method include, alone and in combination with other aspects, said agent killing at least a portion of said cancer cells.

Further aspects of the method include, alone and in combination with other aspects, at least a portion of said cancer cells in step c) undergoing an epithelial-mesenchymal transition.

Further aspects of the method include, alone and in combination with other aspects, the agent inhibiting at least a portion of said cancer cells undergoing said epithelial-mesenchymal transition.

Further aspects of the method include, alone and in combination with other aspects, the method including contacting the co-culture with an agent that inhibits formation of said nodules.

Further aspects of the method include, alone and in combination with other aspects, the method including contacting the co-culture with an agent that inhibits said transmigrating of said membrane.

Tumor cell lines, such as H1975 NSCLC, have mesenchymal-like features, e.g., expressing one or more of vimentin, aSMA, n-cadherin, etc., and lack tight junction formation between cells when growing in culture, after isolation from a primary tumor. Unexpectedly, when mesenchymal-like H1975 NSCLC cells were co-cultured with healthy epithelial cells on chip, NSCLC cells integrated with healthy epithelial cells forming tight-junctions with the healthy cells, in addition to having other indications of being more epithelial-like than mesenchymal-like. These results demonstrate an example of spontaneous/endogenous MET. As shown in FIG. 5D, cancer cells integrated with the healthy epithelial cells forming tight junctions that are a hallmark of epithelial (and epithelial-like) cells. Moreover, surprisingly the integrated MET cancer cells over time transitioned again to become mesenchymal-like (an example of spontaneous/endogenous EMT) in co-cultures on chip with healthy epithelial cells. These EMT cells that returned to expressing morphology of mesenchymal-like cells (i.e., to become more cancer-like cells than healthy cells) then continued to progress to formation of micro-nodules, i.e., simulating solid tumor formation on chip. Thus, unlike co-cultures in plastic culture dishes, the on chip co-cultures simulated the stages of cancer cell progression in addition to the surprising discovery that cancer cells can be induced, at least temporarily, to lose their pathogenic characteristics. In other words, the on chip co-cultures demonstrated the surprising plasticity of cancer cells to become more normal in a normal microenvironment until they return to becoming tumorigenic cells. This discovery, especially when applied to cancer cell movement within organs, and then between organs, provides a new target area of cancer cell treatment. As one example, cancer cell treatments that target MET cells may find use as a primary therapy (i.e., stand-alone therapy) or as a therapy to add to current treatments in order to delay or prevent EMT mediated spreading of cancer cells.

Another discovery was the observation of EMT cancer cells in co-cultures on chips that migrated through the epithelial cell layer into the chip membrane that further continued moving into the endothelial channel. As shown in FIG. 5A, migration of cancer cells from the epithelium into the endothelium was evaluated. Thus, co-cultures on chip provide models for evaluating migration as metastatic potential and actual metastasis. That is to say, that cancer cells, in order to migrate between channels, would need to be at least pre-metastatic in order to invade the vascular channel and then move through the endothelial layer. This model has particular applicability to both the orthotopic model and the metastatic model. In other words, as another orthotopic model, in some embodiments, EMT cancer cells show metastatic potential by migrating from one channel to another, i.e., between channels, e.g., from the epithelial channel into the vascular (endothelial) channel, then return to an adjacent area in the epithelial channel from which it came, or at least one cell may migrate back into the epithelial channel into other areas of the same organ (i.e., within the same chip). Further, in some embodiments, as another orthotopic model, EMT cancer cells show metastatic potential by migrating transversely through the epithelium to in order to spread laterally within the same chip (i.e., same simulated organ). Thus, in some embodiments, cancer cell migration laterally within the epithelial channel is evaluated or measured. In some embodiments, cancer cell migration into the endothelial channel is evaluated. In yet further embodiments, cancer cell migration out of the endothelium back into the epithelial channel is evaluated. In yet further embodiments, cancer cell migration out of the endothelium into the flow area of the channel is evaluated.

Thus, as another metastatic model, in some embodiments, EMT cancer cells migrate between channels, i.e., from the epithelial channel into the vascular (endothelial) channel then continue to migrate into the flow media in order to metastasize into another organ, i.e., into another organ chip.

These and other capabilities of the disclosed embodiments, along with the invention itself, will be more fully understood after a review of the following figures, detailed description, and claims.

### BRIEF DESCRIPTION OF THE DRAWINGS

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

FIG. 1A illustrates an exemplary microfluidic device with a membrane region having cells thereon that may be used with the present invention, in accord with aspects of the present disclosure.

FIG. 1B is a cross-section of the microfluidic device taken along line 1B-1B of FIG. 1A, illustrating a membrane separating the first microchannel and the second microchannel, in accord with aspects of the present disclosure.

FIG. 2A illustrates another exemplary microfluidic device with a membrane region having cells thereon that may be used with the present invention, in accord with aspects of the present disclosure.

FIG. 2B illustrates an exploded view of the microfluidic device of FIG. 2A, illustrating the various portions that combine to form the microfluidic device, in accord with aspects of the present disclosure.

FIG. 2C illustrates a cross-section of the microfluidic device taken along the line 2C-2C of FIG. 2A, illustrating operating channels alongside upper and lower microchannels, in accord with aspects of the present disclosure.

FIG. 2D illustrates a cross-section of the microfluidic device taken along the line 2C-2C of FIG. 2A, illustrating a mechanical force applied to the membrane, in accord with aspects of the present disclosure.

FIG. 3 illustrates a cross-section of a transwell fluidic device, in accord with aspects of the present disclosure.

FIG. 4 illustrates a microfluidic device including cancer cells, in accord with aspects of the present disclosure.

FIG. 5A shows a confocal fluorescence micrograph of a cross-section of a microfluidic device, in accord with aspects of the present disclosure.

FIG. 5B illustrates an immunofluorescence micrograph of an implanted cluster of GFP-labeled NSCLC cancer cells (green), in accord with aspects of the present disclosure.

FIG. 5C shows the quantification of NSCLC tumor cell densities when cultured for up to 1 month after implantation in a differentiated microfluidic chip, in accord with aspects of the present disclosure.

FIG. 5D illustrates cancer cell growth dynamics in various conditions, in accord with aspects of the present disclosure.

FIG. 5E illustrates cancer cell growth dynamics in various conditions, in accord with aspects of the present disclosure.

FIG. 5F illustrates top and cross-sectional confocal fluorescence microscopic views of a non-breathing or static microfluidic device, in accord with aspects of the present disclosure.

FIG. 5G illustrates NSCLC cancer cell growth measured under static conditions, in accord with aspects of the present disclosure.

FIG. 5H illustrates the effects on cancer cell DNA synthesis (EdU incorporation), in accord with aspects of the present disclosure.

FIG. 5I illustrates fluorescence microscopic images of cancer cells, in accord with aspects of the present disclosure.

FIG. 5J illustrates a plot of NSCLC tumor cell growth, in accord with aspects of the present disclosure.

FIG. 5K illustrates a graph of alveolar epithelial cell growth, in accord with aspects of the present disclosure.

FIG. 5L illustrates micrographs showing epifluorescence of EdU incorporation into healthy alveolar epithelium and microvascular endothelium.

FIG. 6A illustrates fluorescence microscopic images of a lung cancer cell cluster, in accord with aspects of the present disclosure.

FIG. 6B illustrates high magnification confocal fluorescence microscopic Z-stack images of GFP-labeled NSCLC cells within the breathing and non-breathing microfluidic devices, in accord with aspects of the present disclosure.

FIG. 6C illustrates a quantification of the invasive behavior shown in FIG. 6B, in accord with aspects of the present disclosure.

FIG. 7A illustrates the sensitivity of tumor cells to various agents, in accord with aspects of the present disclosure.

FIG. 7B illustrates the growth of tumor cells in microfluidic alveolar chips in various culture environments, in accord with aspects of the present disclosure.

FIG. 7C illustrates the levels of various analytes in cancer cells, in accord with aspects of the present disclosure.

FIG. 7D illustrates the production of cytokines, IL-6, IL-8 and VEGF, by NSCLC cancer cells under various conditions, in accord with aspects of the present disclosure.

FIG. 7E illustrates the effect of one or more agents on cancer cells in various environments, in accord with aspects of the present disclosure.

FIG. 8 illustrates a comparison of cancer cell growth in various microfluidic devices, in accord with aspects of the present disclosure.

FIG. 9 illustrates cytokine analysis of medium from transwell co-culture experiments. Epithelial-endothelial-tumor modulation of angiogenic, inflammatory and chemotactic factors was found. Data represents mean of 2 wells over 3 different biological replicates and was normalized with respect to the tumor cell secretion at the density of each corresponding condition; bar represent s.e.m. Significance determined using an unpaired Student's t-test.

FIG. 10 illustrates cytokine analysis of medium from Transwell co-culture experiments for lung specific modulation. Lung specific modulation of angiogenic, inflammatory and chemotactic factors was found. Data represents mean of 2 wells over 2 different biological replicates; bars represent s.e.m. Significance determined using an unpaired Student's t-test.

FIG. 11 illustrates the absence of ZO-1 tight junction protein by immunofluorescence of H1975 monoculture. Cytoplasmic expression in co-culture was observed (bar, 50 μm).

FIG. 12 illustrates a schematic of a liver microfluidic device with primary liver hepatocytes co-cultured in an apical microchannel, liver sinusoidal endothelium lining a basal microchannel, where the blood flow is stimulated, and cancer cells, in accord with aspects of the present disclosure.

FIG. 13A illustrates a top view of an apical channel having colon cancer cells and healthy colon epithelial cells, in accord with aspects of the present disclosure.

FIG. 13B illustrates a cross-section of the apical channel of FIG. 13A, in accord with aspects of the present disclosure.

FIG. 13C illustrates a top view of a basal channel containing human intestinal microvascular endothelial cells (HIMEC), in accord with aspects of the present disclosure.

FIG. 13D illustrates a cross-section of the basal channel of FIG. 13C, in accord with aspects of the present disclosure.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances, the geometry of a microfluidic channel may be configured to control the fluid flow rate through the channel (e.g., increase channel height to reduce shear). Microfluidic channels can be formed of various geometries to facilitate a wide range of flow rates through the channels.

“Channels” are pathways (whether straight, curved, single, multiple, in a network, etc.) through a medium (e.g., silicon) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

As used herein, the phrases “connected to,” “coupled to,” “in contact with,” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid source such as a fluid reservoir. Two components may be coupled to each other even though they are not in direct contact with each other. For example, two components may be coupled to each other through an intermediate component (e.g., tubing or other conduit).

As used herein, the term “co-culture” refers to two or more different cell types being cultured in some embodiments of the devices described herein. The different cell types can be cultured in the same chamber (e.g., first chamber or second chamber) and/or in different chambers (e.g., one cell type in a first chamber and another cell type in a second chamber). For example, in some embodiments, the devices described herein can have hepatocytes in the first chamber and endothelial cells in the second chamber.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. In some embodiments, such markers are proteins, and possess an epitope for antibodies or other binding molecules available in the art, and thus can be monitored by FACs analysis, and immunocytochemistry. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to filtrate particles, ability to migrate under particular conditions, and the ability to differentiate along particular lineages. Markers may be detected by any method available to one of skill in the art, including for example, detection of nucleic acid, e.g. mRNA, e.g. by quantitative PCR.

As used herein, “healthy cells” refers to a cell that exhibits normal cellular division and contains no mutations or alterations in its DNA that make it susceptible or result in a disease state. In contrast, as used herein, “cancer cells” refers to cells which contain DNA mutations or alterations that promote aberrant or uncontrolled cellular divisions and are susceptible or can result in disease states, i.e., tumor formation or metastatic cancer.

The term “differentiated healthy cell” refers to a healthy primary cell that is not in its native form. The term “differentiated cell” also encompasses cells that are partially differentiated, such as multipotent cells (e.g., adult somatic stem cells). In some embodiments, the term “differentiated cell” also refers to a cell of a more specialized cell type derived from a cell of a less specialized cell type (e.g., from an undifferentiated cell or a reprogrammed cell) where the cell has undergone a cellular differentiation process.

In the context of cell ontogeny, the term “differentiate” or “differentiating” is a relative term meaning a “differentiated cell” that has progressed further down the developmental pathway than its precursor cell. Thus in some embodiments, a differentiated cell can differentiate to lineage-restricted precursor cells (such as a epithelial stem cell or a endodermal stem cell), which in turn can differentiate into other types of precursor cells further down the pathway (such as a tissue specific precursor, for example, an alveolar precursor or a hepatocyte precursor), and then to an end-stage differentiated cell, which plays a characteristic role in a certain tissue types, and may or may not retain the capacity to proliferate further.

As used herein, the term “differentiated state” refers to a cell that is partially or fully differentiated to a tissue-specific cell. A fully-differentiated cell can be considered as a cell in a mature state as defined herein. In accordance with some embodiments of the invention, the differentiated cells can form a stratified or pseudo-stratified structure, as defined herein as a differentiated layer. In some embodiments of the invention, the differentiated cells can form a 3-D structure. In some embodiments of the invention, the differentiated cells can form a tissue that is one cell thick, defined herein as a monolayer.

As used herein, the term “stratified structure” refers to cells substantially arranged in more than one layer, e.g., 2 layers, 3 layers, 4 layers, or more.

As used herein, the term “pseudo-stratified structure” refers to cells present in a single layer, but when they are visualized by immunostaining they appear as if they form multiple layers. For example, a pseudostratified epithelium is a type of epithelium that, though comprising only a single layer of cells, has its cell nuclei positioned at different levels, thus creating an illusion of cellular stratification.

As used herein, a cancer cell that “integrates into the tissue layer” is characterized by its presence or physical connection within a tissue layer. For example, the cancer cell that has integrated into a tissue layer is adhered to said tissue layer at at least one cell-cell junction. In one embodiment, a cancer cell can integrate into the top, the bottom, or within the tissue layer. In one embodiment of the invention described herein, the cancer cells express a fluorescent protein which can be detected using microscopy techniques known in the art to assess if the cancer cells have integrated into the tissue layer.

As used herein, a “vascular lumen” on a chip is characterized by a continuous monolayer of endothelial cells that line all four sides of the lower microchannel. The vascular lumen on a chip acts as the interface between the circulating medium and microchannel wall, mimicking the vascular lumen of a blood vessel, which is the interface between the circulating blood and the vessel wall.

As used herein, “inhibiting growth” is characterized by a decrease in cellular division of a healthy cell or a cancer cell, resulting in a decrease of a population thereof. In one embodiment, growth of a cancer cell population is inhibited by at least 1%, by at least 5%, by at least 10%, by at least 20%, by at least 30%, by at least 40%, by at least 50%, by at least 60%, by at least 70%, by at least 80%, by at least 90% by at least 99% or more following contacting said cancer cell population with an agent compared to an untreated cancer cell population. Complete growth inhibition is defined as a 100% inhibition of growth. One skilled in the art can assess inhibition of growth by a number of cellular proliferation assays including, but not limited to, a BrdU incorporation assay, a metabolic cell proliferation assay, or assessing the cell for proliferation markers via immunofluorescence (for example, detecting proliferating cell nuclear antigen (PCNA) protein with a commercially available anti-PCNA antibody from Sigma Aldrich, St. Louis, Mo.).

As used herein, “promoting growth” is characterized by an increase in cellular division of a healthy cell or a cancer cell, resulting in an increase of a population thereof. In one embodiment of the invention, a cancer cell population can be increased by at least 1%, by at least 5%, by at least 10%, by at least 20%, by at least 30%, by at least 40%, by at least 50%, by at least 60%, by at least 70%, by at least 80%, by at least 90% by at least 99% or more following contacting said cancer cell population with an agent compared to an untreated cancer cell population. One skilled in the art can assess an increase in growth by using the proliferative assays described above.

As used herein, “controlling growth” refers to inhibiting growth of a healthy cell, a cancer cell, or a population thereof. In one embodiment, “controlling growth” can refer to slowing of rate growth of a healthy cell, a cancer cell, or a population.

As used herein, “mimics” are meant to resemble, imitate or be similar to at least one or more physiological property (e.g., structure or function) of an organ. In some embodiments, “mimics” substantially similar to at least one or more physiological property of an organ. A “mimic” need not resemble, imitate or be similar to all physiological properties of said organ.

As used herein, “analyzing effects of agents” refers to assessing a characteristic of a cell or population thereof that has been contacted by an agent. Characteristics include, but are not limited to cellular growth, movement in 2D and 3D, cell adhesion, cell size, cell morphology, intracellular pH, DNA expression, gene expression, secretion, cell cycle state, and cellular activity. Assays known in the art that measure cellular growth are described above. Microscopy techniques known in the art can be used to analyze cellular movement in 2D and 3D, cell size, and cell morphology. Intracellular pH can be measured using pHrodo dye, a fluorogenic dye that increases in fluorescence intensity at a lower pH; pHrodo dye is commercially available from ThermoFisher Scientific (Waltham, Mass.). Cell cycle state, cell adhesion, and cellular activity can be assessed by probing a cell with cell cycle-, adhesion-, or activity-specific markers known in the art and imaging the cell to determine the presence or absence of said marker. Secretion can be assessed using techniques known in the art, for example probing the medium a cell or population thereof is maintained in using western blot analysis to detect a secreted protein.

As used herein, “measuring a response” refers to comparing a characteristic of a cell or population thereof that has been contacted by an agent to said characteristic of an identical cell or population thereof that has not been contacted by an agent. For example, the cell morphology of a cancer cell population that has been contacted by an agent is assessed and compared to an identical cancer cell population that has not been contacted by an agent. A change in morphology between these populations is the measured response. In one embodiment, a characteristic of a cell or population thereof that has been contacted by an agent is compared to a non-identical cell or population thereof that has not been contacted by an agent. In one embodiment, a characteristic of a cell or population thereof that has been contacted by an agent is compared to said characteristic of said cell or population thereof prior to contact with an agent or at various time points while being contacted by an agent. For example, the cell morphology of a cancer cell population can be assessed prior to being contacted by an agent, 12 hours after contact by an agent, and 24 hours after contacted by an agent. The cell morphology at these three time points would be compared.

As used herein, “analysis of molecular level modulation of drug action” is characterized by comparing changes to various molecular aspects of the drug action for an agent when exposed to the invention described herein. In some embodiments, one or more devices described herein can be used in combination with a pharmacokinetic (PK) model, a pharmacodynamic (PD) model, or a PK-PD model to quantitatively analyze the effect of an agent to be tested. The term “pharmacokinetics” is used herein in accordance with the art, and refers to the study of the action of agents, e.g., drugs, in the first structure and/or second structure, for example, the effect and duration of drug action, the rate at which they are absorbed, distributed, metabolized, and eliminated by the first structure and/or second structure etc. (e.g. the study of a concentration of an agent, e.g., a drug, in the serum of a patient following its administration via a specific dose or therapeutic regimen). The term “pharmacodynamics” is used in accordance with the art, and refers to the study of the biochemical and physiological effects of an agent, e.g., a drug, on a subject's first structure and/or second structure or on microorganisms such as viruses within or on the first structure and/or second structure, and the mechanisms of drug action and the relationship between drug concentration and effect (e.g. the study of a pathogen, e.g., a virus, present in a patient's plasma following one or more therapeutic regimens). Methods for PK-PD modeling and analysis are known in the art. See, e.g., Bonate, P. L. (2006). Pharmacokinetic-Pharmacodynamic Modeling and Simulation. New York, Springer Science & Business Media; Gabrielsson, J. and D. Weiner (2000); and Phannacokinetic and Pharmacodynamic Data Analysis: Concepts and Applications. Stockholm, Swedish Pharmaceutical Press. For example, a PK model can be developed to model a microphysiological system comprising a plurality of the devices described herein, wherein each device can model a different tissue (for example one for lung and one for liver) that can produce an effect (e.g., absorption, metabolism, distribution and/or excretion) on an agent to be administered. To construct a PK model for a device described herein, mass balance equations describing the flow in, flow out, and metabolism of an agent can be set up for each first chamber and second chamber. A PD model can be integrated into each device described herein, describing the kinetics of potential cell response (e.g., inflammation, cytokine release, ligand binding, cell membrane disruption, cell mutation and/or cell death) in each device that mimics a tissue or an organ. This in vitro/in silico system, combining one or more devices described herein with an integrated PK-PD modeling approach, can be used to predict drug toxicity in a more realistic manner than conventional in vitro systems. In some embodiments, one or more of the devices described herein can be used to quantify, estimate or gauge one or more physical-chemical, pharmacokinetic and/or pharmacodynamic parameters. Various physical-chemical, pharmacokinetic and pharmacodynamic parameters are known in the art, including, for example, the ones discussed in the aforementioned references. Exemplary physical-chemical, pharmacokinetic and pharmacodynamic parameters include, but are not limited to, permeability, log P, log D, volume of distribution, clearances (including intrinsic clearances), absorption rates, rates of metabolism, exchange rates, distribution rates and properties, excretion rates, IC50, binding coefficients, etc.

Microfluidic devices and methods are disclosed that provide for the ability to create orthotopic microenvironments. The orthotopic microenvironments obtained according to aspects of the present disclosure provide the ability to control cancer cells, such as human cancer cells, in an organ-relevant microenvironment to be in a non-growing, dormant state. Such a non-growing, dormant state within an orthotopic microenvironment provides the ability to study the cancer cell dormancy, which is one of the key challenges that must be overcome to prevent cancer recurrence in patients in remission or with residual disease. Control over the cancer cells within the orthotopic microenvironments is obtained by controlling cues elicited by healthy cells within the orthotopic microenvironment, such as epithelial and endothelial cells and tissues, as well as by controlling mechanical and/or shearing forces applied to the cancer cells. Thus, the orthotopic microenvironments established using the microfluidic devices according to the present disclosure mimic the unique growth patterns that are observed in human patients with the cancer in vivo. Further, control over the cancer cells provides for new ways of investigating the effects of drugs and other agents on the cancer cells. Such ability provides for investigating why it is so difficult to eradicate residual cancer cells, also referred to as persister cells, which can lead to development of new forms of drug resistance mitigation in the future. The orthotopic microenvironments provided by the microfluidic devices provide the ability to uncover new insights into mechanisms of cancer control, and to facilitate discovery of novel drug targets and anti-cancer therapeutics.

The functionality of cells and tissue types (and even organs) can be implemented in one or more microfluidic devices or “chips” that enable researchers to study these cells and tissue types outside of the body while mimicking much of the stimuli and environment that the tissue is exposed to in vivo. It can also be desirable to implement these microfluidic devices into interconnected components that can simulate groups of organs or tissue systems. Preferably, the microfluidic devices can be easily inserted and removed from an underlying fluidic system that connects to these devices in order to vary the simulated in-vivo conditions and organ systems.

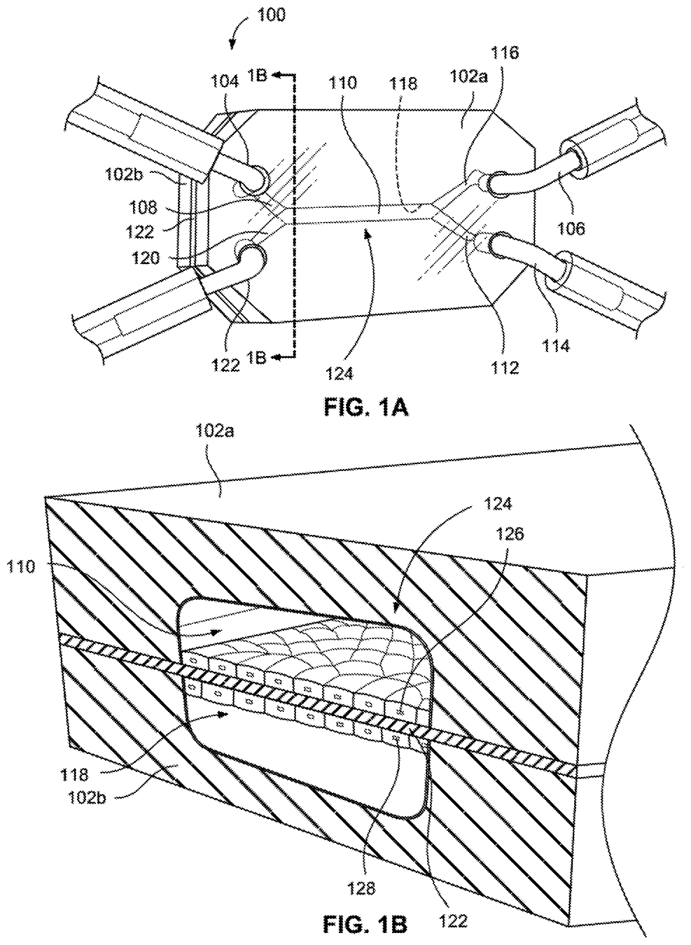

FIGS. 1A and 1B illustrate one type of an organ-on-chip (“OOC”) device 100, also referred to as a microfluidic device 100. The microfluidic device 100 includes a body 102 that is typically comprised of an upper body segment 102a and a lower body segment 102b. The upper body segment 102a and the lower body segment 102b can be made of a flexible material or an inflexible material polymeric material, such as polydimethylsiloxane (PDMS). The upper body segment 102a includes a first fluid inlet 104 and a second fluid inlet 106. A first fluid path for a first fluid includes the first fluid inlet 104, a first seeding channel 108, an upper microchannel 110, an exit channel 112, and a first fluid outlet 106. A second fluid path for a second fluid includes the second fluid inlet 106, a first seeding channel 116, a lower microchannel 118, an outlet channel 120, and a second fluid outlet 122.

As seen best in FIG. 1B, a membrane 122 extends between the upper body segment 102a and the lower body segment 102b. The membrane 122 is preferably an inert, polymeric, micro-molded membrane having uniformly distributed pores with sizes in the range large enough to allow cells to pass therethrough. The thickness of the membrane 122 is generally in the range of about of about 5 μm to about 100 μm, such as about 10 μm. Preferably, the membrane 122 is made of semi-porous polyester (PET) or PDMS and is about 25 to 50 μm thick, with about 9 μm in diameter pores. With the 9 μm pores cells, and particularly cancer cells, are able to migrate through the membrane 122 to mimic migration found in vivo. In one or more embodiments, the membrane 122 can be plasma treated and coated with an extracellular matrix (ECM). The ECM can contain, for example, 0.5 mg/mL of laminin, 1 mg/mL of fibronectin, and 3.2 mg/mL1 of collagen type I. In one or more embodiments, the membrane 122 can be capable of stretching and expanding in one or more planes to simulate the physiological effects of expansion and contraction forces that are commonly experienced by cells.

The membrane 122 separates the upper microchannel 110 from the lower microchannel 118 in an active region 124, which includes a bilayer of cells in the illustrated embodiment. In particular, a first cell layer 126 is adhered to a first side of the membrane 122, while a second cell layer 128 is adhered to a second side of the membrane 122. The first cell layer 126 may include the same type of cells as the second cell layer 128. Alternatively, the first cell layer 126 may include a different type of cell than the second cell layer 128. While a single layer of cells is shown for the first cell layer 126 and the second cell layer 128, the first cell layer 126 and the second cell layer 128 may include multiple cell layers or groupings of cells. Further, while the illustrated embodiment includes a bilayer of cells on the membrane 122, the membrane 122 may include only a single cell layer disposed on one of its sides or multiple layers and/or grouping of cells on one or both sides. Further, in one or more embodiments, the membrane 122 can be coated with at least one attachment molecule that supports adhesion of cells to the membrane, such as healthy cells and/or cancer cells, as discussed below.

The microfluidic device 100 is configured to simulate a biological function that typically includes cellular communication between the first cell layer 126 and the second cell layer 128, as would be experienced in vivo within organs, tissues, etc. Depending on the application, the membrane 122 is designed to have at least a partial porosity to permit the migration of cells, particulates, media, proteins, and/or chemicals between the upper microchannel 110 and the lower microchannel 118. The working fluids within the microchannels 110, 118 may be the same fluid or different fluids. As one example, the microfluidic device 100 simulating a lung may have air as the fluid in one microchannel and a liquid simulating blood in the other microchannel. When developing the cell layers 126 and 128 on the membrane 122, the working fluids may be a tissue-culturing fluid.

In one or more embodiments, the upper microchannel 110 can have a height of about 2000 μm or less and a width about 2000 μm or less. In one or more embodiments, the lower microchannel 118 can have dimensions that are the same as the upper microchannel 110. Alternatively, the lower microchannel 118 can have different dimensions, such as a height of about 200 μm or less and a width about 2000 μm or less. The active region 124 defined by overlap of the upper and lower microchannels 110, 118 can have a length of about 2 cm or less. The microfluidic device 100 can include an optical window that permits viewing of the fluids, media, particulates, etc. as they move across the first cell layer 126 and the second cell layer 128. Alternatively, the microfluidic device 100 can be formed of an optically transparent material that permits viewing of the fluids, media, particulates, etc. Various image-gathering techniques, such as spectroscopy and microscopy, can be used to quantify and evaluate the effects of the fluid flow in the microchannels 110, 118, as well as cellular behavior and cellular communication through the membrane 122. More details on the microfluidic device 100 can be found in, for example, U.S. Pat. No. 8,647,861, which is owned by the assignee of the present application and is incorporated by reference in its entirety.

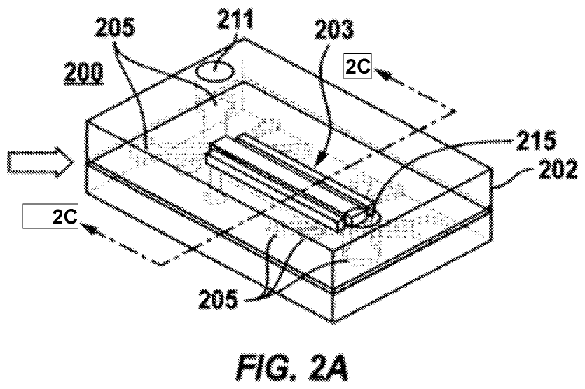

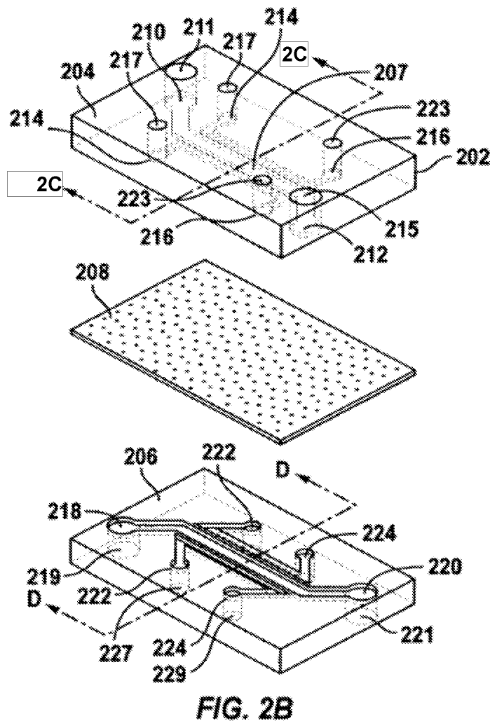

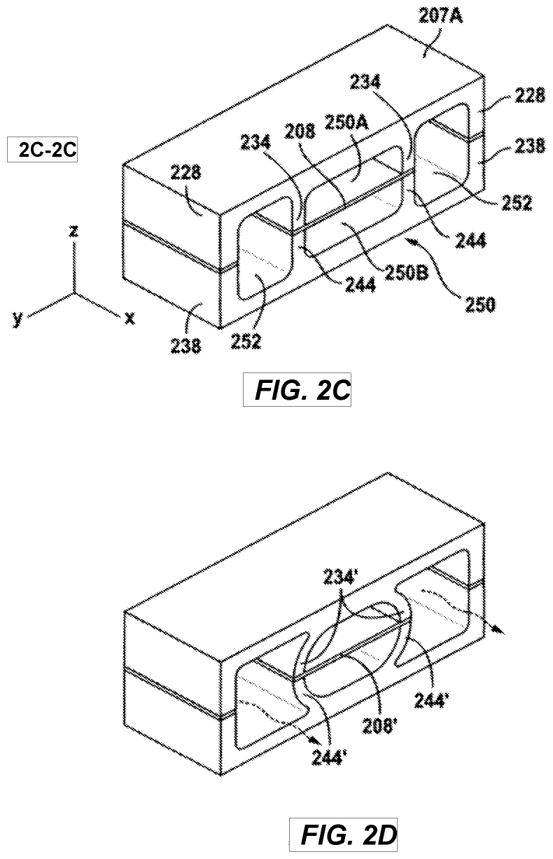

FIGS. 2A-2D illustrate another type of 00C device 200, also referred to as a microfluidic device 200. FIG. 2A shows that the device 200 includes a body 202 having a branched microchannel design 203. The body 202 is preferably made of a flexible biocompatible polymer, including but not limited to PDMS or polyimide. Alternatively, the body 202 can be made of non-flexible materials like glass, silicon, hard plastic, and the like.

FIG. 2B illustrates an exploded view of the microfluidic device 200. In particular, the device 200 is comprised of the body 202, which is comprised of a first body portion 204 and a second body portion 206, and an at least partially porous membrane 208 configured to be mounted between the first and second body portions 204, 206 when the portions 204, 206 are mounted to one another to form the overall body 202. The body 202 further defines an upper microchannel 250A that is separated from a lower microchannel 250B by the membrane 208. Although referred to separately as the upper microchannel and the lower microchannel 250A, 250B, in some aspects the upper microchannel and the lower microchannel 250A, 250B together are referred to as the main microchannel 250, such that the membrane separates the main microchannel 250 into the upper microchannel 250A and the lower microchannel 250B. The membrane 208 allows for the investigation of cell behavior and/or the monitoring and/or passage of gases, chemicals, molecules, particulates, and cells.

As shown in FIG. 2B, the first body portion 204 includes one or more inlet fluid ports 210 preferably in communication with one or more corresponding inlet apertures 211 located on an outer surface of the body 202. The microfluidic device 200 is preferably connected to a fluid source (not shown) via the inlet aperture 211 in which fluid travels from the fluid source into the microfluidic device 200 through the inlet fluid port 210.

Additionally, the first body portion 204 includes one or more outlet fluid ports 212 preferably in communication with one or more corresponding outlet apertures 215 on the outer surface of the body 202. In particular, fluid passing through the microfluidic device 200 exits the microfluidic device 200 to, for example, a fluid collector (not shown) or other appropriate component via the corresponding outlet aperture 215. The microfluidic device 200 may be set up such that the fluid port 210 is an outlet and fluid port 212 is an inlet. Although the inlet and outlet apertures 211, 215 are shown on the top surface of the body 202, one or more of the apertures may be located on one or more sides of the body 202.

The inlet fluid port 210 and the outlet fluid port 212 are in communication with the upper microchannel 250A such that fluid can dynamically travel from the inlet fluid port 210 to the outlet fluid port 212 via the upper microchannel 250A, independently of the lower microchannel 250B. It is also contemplated that the fluid passing between the inlet and outlet fluid ports 210, 212 may be shared between the upper and lower microchannels 250A and 250B. In either embodiment, characteristics of the fluid flow, such as flow rate and the like, passing through the upper microchannel 250A is controllable independently of fluid flow characteristics through the lower microchannel 250B and vice versa.

The first body portion 204 can include one or more pressure inlet ports 214 and one or more pressure outlet ports 216, in which the inlet ports 214 are in communication with apertures 217 and the outlet ports 216 are in communication with corresponding apertures 223 located on the outer surface of the device 100. However, the microfluidic device 200 may be set up such that the pressure port 214 is an outlet and pressure port 216 is an inlet. Further, although the pressure apertures 217, 223 are shown on the top surface of the body 202, one or more of the pressure apertures 217, 223 can be located on one or more side surfaces of the body 202. The inlet ports 214 are in fluidic communication to the outlet ports 216 through operating channels 252 (FIGS. 2C and 2D).

In operation, one or more pressure tubes (not shown) can provide positive or negative pressure to the device via the apertures 217. Additionally, pressure tubes can be connected to the microfluidic device 200 to remove the pressurized fluid from the outlet port 216 via the apertures 223.

The second outer body portion 206 can includes one or more inlet fluid ports 218 and one or more outlet fluid ports 220. The inlet fluid port 218 is in communication with aperture 219 and the outlet fluid port 220 is in communication with aperture 221, whereby the apertures 219 and 221 are located on the outer surface of the second outer body portion 206. Although the inlet and outlet apertures 219, 221 are shown on the surface of the body 202, one or more of the apertures may be alternatively located on one or more sides of the body.

As with the first outer body portion 204 described above, one or more fluid tubes (not shown) connected to a fluid source (not shown) are preferably coupled to the aperture 219 to provide fluid to the microfluidic device via port 218. Additionally, fluid exits the microfluidic device 200 via the outlet port 220 and outlet aperture 221 to a fluid reservoir/collector or other component (not shown). It should be noted that the device 200 may be set up such that the fluid port 218 is an outlet and fluid port 220 is an inlet.

The second outer body portion 206 includes one or more pressure inlet ports 222 and one or more pressure outlet ports 224, in which the inlet ports 222 are in communication with apertures 227 and the outlet ports 224 can be in communication with apertures 229, whereby apertures 227 and 229 are located on the outer surface of the second portion 206. Although the inlet and outlet apertures are shown on the bottom surface of the body 202, one or more of the apertures may be alternatively located on one or more sides of the body. Pressure tubes connected to a pressure source are preferably engaged with ports 222 and 224 via corresponding apertures 227 and 229. It should be noted that the device 200 may be set up such that the pressure port 222 is an outlet and fluid port 224 is an inlet. The inlet ports 222 are in fluidic communication to the outlet ports 224 through operating channels 252 (FIGS. 2C and 2D). However, in one or more embodiments, the second body portion 206 can lack the inlet ports 222, the outlet ports 224, and the apertures 227, 229.

The membrane 208 is mounted between the first portion 204 and the second portion 206, whereby the membrane 208 is located within the body 202 of the device 200. As described above, the membrane 208 is a made of a material at least partially having a plurality of pores or apertures therethrough, whereby molecules, cells, fluid, or any media is capable of passing through the membrane 208 via one or more pores in the membrane 208. The membrane 208 can be made of the same or different material as the body 202. In some aspects, the membrane 208 can be made of a material that allows the membrane 208 to undergo stress and/or strain in response to mechanical forces applied to the membrane 208. In one or more embodiments, the mechanical forces can be applied based on pressure differentials present between the main microchannel 250 and the operating microchannels 252. Alternatively, the porous membrane 208 can be relatively inelastic, in which the membrane 208 undergoes minimal or no movement while media is passed through one or more of the upper and lower microchannels 250A, 250B and cells organize and move between the upper and lower microchannels 250A, 250B via the porous membrane.

FIG. 2C illustrates a cross-section of the membrane 208 within the body 202 through the line 2C-2C in FIG. 2A. In particular, FIG. 2C illustrates the first portion 204 and the second portion 206 mated to one another whereby side walls 228, 238 as well as channel walls 234, 244 form the upper and lower microchannels 250A and 250B, separated by the membrane 208, and the operating microchannels 252. The main microchannel 250 and operating microchannels 252 are separated by the walls 234, 244 such that fluid is not able to pass between the channels 250, 252.

The membrane 208 is preferably positioned in the center of the central microchannel 250 and is oriented along a plane parallel to the x-y plane shown in FIG. 2C. It should be noted that although one membrane 208 is shown separating the upper and lower microchannels 250A and 250B, more than one membrane 208 may be configured within the body 202. In addition, the membrane 208 can be sandwiched in place by channel walls 234, 244 during formation of the device. Further, although the membrane 208 is shown midway through the central microchannel 250, the membrane 208 may alternatively be positioned vertically off-center, thus making one of the upper or lower microchannel 250A, 250B larger in volume or cross-section than the other.

With regard to the membrane 208, a pressure differential may be applied within the microfluidic device 200 to cause relative continuous expansion and contraction of the membrane 208 along the x-y plane. In particular, one or more pressure sources preferably apply pressurized fluid (e.g., air) along the one or more operating microchannels 252, whereby the pressurized fluid in the microchannels 252 creates a pressure differential on the microchannel walls 234, 244. The membrane 208 may have elasticity depending on the type of material that it is made of.

In the embodiments shown in FIGS. 2C and 2D, the pressurized fluid is a vacuum or suction force that is applied only through the operating microchannels 252. The difference in pressure caused by the suction force against the microchannel walls 234, 244 causes the walls 234, 244 to bend or bulge outward toward the sides of the device 228, 238 (see FIG. 2D). Considering that the membrane 208 is mounted to and sandwiched between the walls 234, 244, the relative movement of the walls 234, 244 thereby causes the opposing ends of the membrane to move along with the walls to stretch (shown as 208′ in FIG. 2D) along the membrane's plane. This stretching mimics the mechanical forces experienced by a tissue-tissue interface, for example, in the lung alveolus during breathing, and thus provides the important regulation for cellular self-assembly into tissue structures and cell behavior.

When the negative pressure is no longer applied (and/or positive pressure is applied to the operating channels), the pressure differential between the operating channels 252 and the upper and lower microchannels 250A and 250B decreases and the channel walls 234, 244 retract elastically toward their neutral position (as in FIG. 2C). During operation, the negative pressure is alternately applied in timed intervals to the microfluidic device 200 to cause continuous expansion and contraction of the membrane 208 along its plane, thereby mimicking operation of the tissue-tissue interface of the living organ within a controlled in vitro environment. As will be discussed, this mimicked organ operation within the controlled environment allows monitoring of cell behavior in the tissues, as well as passage of molecules, chemicals, particulates and cells with respect to the membrane and the upper and lower microchannels 250A, 250B.

It should be noted that the term pressure differential in the present specification relates to a difference of pressure on opposing sides of a particular wall between the upper and lower microchannels 250A and 250B and one or more of the outer operating channels 250. It is contemplated that the pressure differential may be created in a number of ways to achieve the goal of expansion and/or contraction of the membrane 208. As stated above, a negative pressure (i.e., suction or vacuum) may be applied to one or more of the operating channels 252. Alternatively, it is contemplated that the membrane 208 is pre-loaded or pre-stressed to be in an expanded state by default such that the walls 234, 244 are already in the bent configuration, as show in FIG. 2D. In this embodiment, positive pressure applied to one or more of the operating channels 252 will create the pressure differential which causes the walls 234 and/or 244 to move inward toward the upper and lower microchannels 250A and 250B (see in FIG. 2C) to contract the membrane 208.

It is also contemplated, in another embodiment, that a combination of positive and negative pressure is applied to one or more operating microchannels 252 to cause movement of the membrane 208 along its plane in the upper and lower microchannels 250A and 250B. In any of the above embodiments, it is desired that the pressure of the fluid in the one or more operating channels 252 be such that a pressure differential is in fact created with respect to the pressure of the fluid(s) in one or more of the upper and lower microchannels 250A and 250B to cause relative expansion/contraction of the membrane 208. For example, fluid may have a certain pressure may be applied within the top central microchannel 250A, whereby fluid in the bottom central microchannel 250B may have a different pressure. In this example, pressure applied to the one or more operating channels 252 must take into account the pressure of the fluid in either or both of the central microchannels 250A, 250B to ensure desired expansion/contraction of the membrane 208.

In one or more embodiments, it is possible for a pressure differential to exist between the top and bottom microchannels 250A, 250B to cause at least a portion of the membrane 208 to expand and/or contract vertically in the z-direction in addition to expansion/contraction along the x-y plane.

In one or more embodiments, the expansion and retraction of the membrane 208 preferably applies mechanical forces to the adherent cells and ECM that mimic physiological mechanical cues that can influence transport of chemicals, molecules particulates, and/or fluids or gas across the tissue-tissue interface, and alter cell physiology. It should be noted that although pressure differentials created in the device preferably cause expansion/contraction of the membrane, it is contemplated that mechanical means, such as micromotors or actuators, may be employed to assist or substitute for the pressure differential to cause expansion/contraction of the cells on the membrane to modulate cell physiology.

Although not shown, cell layers can be formed on the surfaces of the membrane 208, such as the cell layers 126 and 128 in the microfluidic device 100. Similar to above, a first cell layer can be formed on a first side of the membrane 208, and the first cell layer can include the same type of cells or multiple different types of cells. A second cell layer can be formed on a second side of the membrane 208, and the second cell layer can include the same type of cells or multiple different types of cells. Further, the first cell layer may include a different type of cell than the second cell layer. The first and second cell layer also can be formed of a single layer of cells or multiple cell layers or groupings of cells. Further, the membrane 208 may include only a single cell layer disposed on one of its sides or multiple layers and/or grouping of cells on one or both sides.

While aspects of the present disclosure can be applied to the microfluidic devices 100 and 200, the microfluidic devices 100 and 200 are merely exemplary. Aspects of the present disclosure can be applied to other microfluidic devices than microfluidic devices 100 and 200 without departing from the spirit and scope of the present disclosure. For example, the microfluidic devices can have more than one upper and/or lower microchannel, more or less than two operating channels 252, etc. Thus, the microfluidic devices 100 and 200 are not meant to be limiting and merely are for describing aspects of the present disclosure.

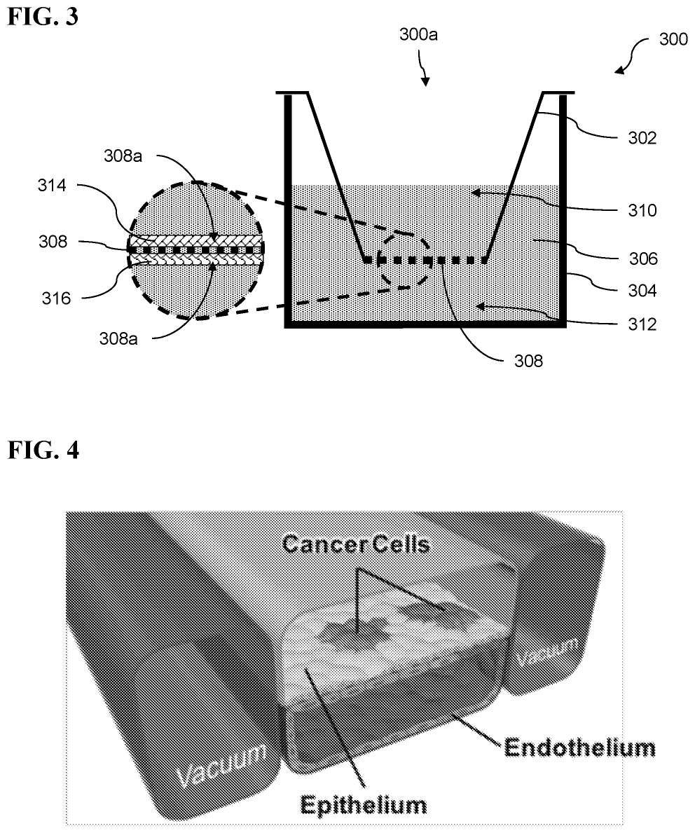

FIG. 3 illustrates a transwell 300, which is another type of device to which aspects of the present disclosure can be applied. The transwell 300 is formed of a transwell insert 302 that sits within a well 304. The well 304 can contain material 306, such as various fluids and solids (e.g., particulates, cells, etc.). The transwell insert 302 includes a membrane 308 near the bottom thereof. The membrane 308 can be similar or identical to any membrane disclosed herein, such as the membrane 122 discussed above. The membrane 308 divides the well 304 into a first chamber 310, above the membrane 308 and within the transwell insert 302, and a second chamber 312, below the membrane 308 and within the well 304 but outside of the transwell insert 302.

Referring to the expanded view of the membrane 308 in FIG. 3, a first cell layer 314 can be on a first side 308a of the membrane 308 facing the first chamber 310, and a second cell layer 316 can be on a second side 308b of the membrane 308 facing the second chamber 312. The membrane 308 permits material 306 (e.g., fluid, cells, etc.) to pass between the first and second chambers 312 and 314. In one or more embodiments, the first chamber 310, the second chamber 312, or both can be lined within a cell layer (not shown), either in addition to a cell layer on the membrane 308, or in the alternative to a cell layer on the membrane 308. Material 306 can be added or removed from the first and second chambers 310 and 312 via the top 300a of the transwell 300.

The cell layers described above on the membranes, such as the cell layers 126 and 128, can include various types of cells. The cells can be healthy cells and cancer cells. Healthy cells are normal cells found within the body, with a normal organ-specific microbiome. In general, and for the study of cancer, healthy cells are cells that are not cancerous. Further, the healthy cells used can be any type of healthy cell depending on the microenvironment being analyzed. For example, although the healthy cells disclosed herein are primarily related to the lung, the healthy cells can be related to any type of healthy cell in the body, such as cells from the liver, brain, kidney, stomach, heart, pancreas, bladder, colon, intestine, skin, or any other organ or system within the body, including human and non-human bodies (e.g., non-human animals).

Although the present disclosure focuses primarily on healthy cells, such as epithelial and endothelial cells, used to form the cell layers on the membranes of microfluidic devices, in addition to the cancer cells, other cells can be added to the microfluidic devices to further capture the microenvironments found in vivo. For example, additional cells that can be added to the microfluidic devices include cancer stromal cells or other stromal cells (e.g., fibroblasts, pericytes, astrocytes), immune cells (e.g., neutrophils, macrophages, dendritic cells, B and T lymphocytes), or any other relevant type of cell.

With the microfluidic device 100 as an example, in one or more embodiments, the cell layer 126 can be formed of epithelial cells, which together form an epithelium layer as the cell layer 126. The cell layer 128 can be formed of endothelial cells, which together form an endothelium layer as the cell layer 128. In such a configuration, the upper and lower microchannels 110 and 118 can correspond to, for example, an alveolus within the lung. The upper microchannel 110 corresponds to the airside and the lower microchannel 118 corresponds to the blood side. The epithelial and endothelial cells represent healthy cells (e.g., non-cancerous cells). However, any cell type can be added to the microfluidic devices of the present disclosure. Such healthy cells can be various epithelial and endothelial cells. In one or more embodiments, the healthy cells can be primary cells, mammalian primary cells, human primary cells, human primary epithelial cells, human primary airway epithelial cells, primary endothelial cells, primary stromal cells, primary lung cells, lung alveolar or airway epithelial cells, human primary alveolar epithelial cells, human lung microvascular endothelial cells, or any other type of healthy cell.

FIG. 4 shows a microfluidic device as described in the preceding paragraph with the addition of cancer cells. The addition of cancer cells to the microfluidic device permits the study of cancer within a microenvironment modeled by the microfluidic device. The cancer cells can be any type of cancer cell, such as primary cancer cells, human primary cancer cells, cancer cells from an established cancer cell line, such as an established cancer line from human tissue, lung cancer cells, non-small cell lung cancer (NSCLC) cells, NSCLC adenocarcinoma cells, etc. The specific type of cancer cells used can depend on the specific microenvironment being analyzed, such as the lungs, kidneys, liver, brain, stomach, heart, colon, etc. In the case of lung cancer, the cancer cells can be, for example, NSCLC cells, such as NSCLC adenocarcinoma cells.

As shown in FIG. 4, the cancer cells can be included on one side of the membrane. As specifically illustrated, the cancer cells can be on the side of the membrane corresponding to the epithelial cells or a differentiated epithelial cell layer. The cancer cells can be integrated within, above, and/or below the differentiated epithelial cell layer, such as on the epithelial cell layer, integrated into the epithelial cell layer, within one or more pores of the membrane covered by the epithelial cell layer, or a combination thereof. As an alternative to being on the membrane within the upper microchannel, the cancer cells can be on the membrane within the lower microchannel, within the pores between the microchannels, or a combination thereof. The addition of the cancer cells generates an orthotopic model representative of an in vivo microenvironment, which, in the case of FIG. 4, is inside the lungs for lung cancer.

Although cancer cells can be included with healthy cells on a surface of the membrane, how the cancer cells are added to the membrane affects the growth of the cancer cells. The effect on the growth of the cancer cells in turn affects how the orthotopic model mimics in vivo conditions. Thus, the process of adding the cancer cells to the membrane provides for control over the cancer cells that can be tied to specific investigations. For example, the addition of the cancer cells can prohibit cell growth, which can mimic certain situations found in vivo, such as persister cancer cells.

The process of adding the cancer cells to the membrane, such as through seeding, plating, or injection, followed by the culturing of the cancer cells, affects the orthotopic model. The cancer cell density, the ratio of cancer cells to healthy cells, and the type of healthy cells surrounding the cancer cells or forming the cellular layer within which the cancer cells are integrated also affect the orthotopic model. By altering the foregoing characteristics, cancer cell growth can be controlled to mimic certain cancer cell growth in vivo, such as conditions where cancer cell growth is promoted or conditions where cancer cell growth is inhibited, that have previously not been obtainable with other models.

In one or more embodiments, the cancer cells can be added to the membrane within a microchannel, such as the upper and/or lower microchannel, by seeding both cancer cells and healthy cells at the same time, or by seeding the cancer cells onto the membrane prior to a differentiated cell layer of the healthy cells being formed. In such a process, a seeding medium can include both healthy cells and cancer cells to seed the healthy cells simultaneously with the cancer cells. Alternatively, one seeding medium can separately include the healthy cells, and another seeding medium can separately include the cancer cells. The seeding medium containing the cancer cells can be perfused through the desired microchannel prior to perfusing the culture medium with the healthy cells. Alternatively, the seeding medium containing the cancer cells can be perfused through the desired microchannel simultaneously with the seeding medium containing the healthy cells. A single seeding medium containing both cancer cells and healthy cells can be flowed through the microchannel. According to these approaches, the cancer cells are seeded onto the membrane prior to a differentiated cell layer of the healthy cells forming. Allowing the cancer cells to seed on the membrane prior to differentiation of the healthy cell layer promotes subsequent growth of the cancer cells. In contrast, and as described below, seeding the cancer cells on the membrane after a differentiated cell layer of healthy cells is formed can inhibit growth of the cancer cells.

The cell density of the cancer cells seeded on the membrane also can control the orthotopic model. In particular, control of the cell density of the cancer cells controls the subsequent growth of the cancer cells during subsequent culturing. Cancer cell densities seeded within the range of about 100 cells/cm² to about 10,000 cells/cm² promotes subsequent growth of the cancer cells in the orthotopic model. In contrast, cancer cell densities outside of the foregoing range can inhibit cancer cell growth during subsequent cell culturing.

The ratio of cancer cells to healthy cells seeded on the membrane also controls the orthotopic model. In particular, control of the ratio of the cancer cells to the healthy cells controls the growth of the cancer cells during subsequent culturing. Cancer cells seeded at a ratio of healthy cells to cancer cells of about 25:1 to about 500:1 promotes subsequent growth of the cancer cells in the orthotopic model. In contrast, ratios of cancer cells to healthy cells outside of the foregoing ratio range inhibits cancer cell growth during subsequent cell culturing.

By way of example, an orthotopic model of human NSCLC cells representative of the in vivo lung microenvironment was created. The cancer cells used were H1975 human NSCLC adenocarcinoma cells. The H1975 NSCLC adenocarcinoma cells were infected with lentivirus containing the transgene integration, (CMV) to Luciferase=>(SV40)=>eGFP-IRES-puro (GeneCopoeia™), according to the manufacturer's protocol with 5 μg/mL polybrene at 4° C. for 1 hour. The healthy cells used were primary lung alveolar or small airway epithelial cells. In some aspects, the NSCLC cells can be engineered to express high levels of green fluorescent protein (GFP). In particular, puromycin (1 μM) was included in cancer cell cultures for 2-3 passages, after which the cancer cells exhibited stable high levels of GFP expression for multiple passages (>1 month). The fluorescent protein aids in optically quantifying cancer cell growth and invasion, as further discussed below.

The cancer cells were seeded on an upper surface of a membrane facing an upper microchannel at a cell density of about 3200 cells/cm². However, in some aspects of the present disclosure, the cell density can be about 100 to about 10,000 cells/cm², as described above. The cancer cells also were seeded on the membrane at a healthy cell to cancer cell ratio of about 100:1. However, in some aspects of the present disclosure, the ratio can between about 25:1 and about 500:1 of healthy cells to cancer cells, as described above. The cancer cells were first seeded on the membrane, followed by seeding of the healthy cells. Seeding the cancer cells prior to differentiation of the healthy cells caused the cancer cells to integrate into the resulting epithelial cell layer during the tissue differentiation process, which aided in promoting cell growth during culturing. After seeding, the seeding medium was removed from the upper microchannel and replaced with air, resulting in an air interface between the resulting epithelium.

By way of another example, GFP-labeled NSCLC cells were injected with a syringe into the upper microchannel of a microfluidic device. About 530 NSCLC cells were injected within 35 μl epithelial growth medium at a cell density of 3200 cells/cm². The NSCLC cells were allowed to attach to the membrane for 3 hours under static conditions at 37° C. before the epithelial growth medium was removed gently with a syringe. Subsequently, primary small airway epithelial cells were injected. About 33,000 primary small airway epithelial cells were injected in 35 μL of epithelial growth medium at a density of 2×10⁵ cells/cm² into the upper microchannel.

The microfluidic device was cultured statically and the culture medium was removed from the upper microchannel after to create an air-liquid (ALI) interface. An ALI medium supplemented with 50 ng/mL retinoic acid was then perfused through the lower microchannel at 60 μL/hr. After the airway epithelial cells were allowed to differentiate for approximately 2 weeks, the microfluidic devices were taken off flow, and primary human lung microvascular endothelial cells were seeded in the lower microchannel of the microfluidic device to line the lower microchannel with an endothelial cell layer. About 3.3×10⁵ cells were seeded with 35 μL of seeding medium and a density of about 2×10⁶ cells/cm². The microfluidic device was subsequently cultured in an incubator upside down for 2 hours under static conditions before being returned to its normal right side up orientation for another hour to ensure endothelial cell coverage of all four walls of the lower microchannel. The microfluidic device was then perfused at 60 μL/hr through the lower microchannel for the remainder of culturing.

By way of another example, applied to non-static microfluidic devices, such as the microfluidic device 200, the microfluidic device was plasma treated, then immediately exposed to 10% aminopropyltrimethsiloxane (APTMS) in pure ethanol for 10 minutes, washed 3 times with ethanol, dried in an 80° C. oven overnight, and then coated with the an ECM mixture. Primary human lung microvascular endothelial cells were plated the same way as discussed above, except that these cells were plated first with endothelial growth medium. The GFP-labeled NSCLC tumor cells and primary alveolar epithelial cells were then plated using the same method as described above. The following day, the upper microchannel was perfused with epithelial growth medium containing 1 μM dexamethasone and flushed manually for 2-3 days, after which the medium in the upper microchannel was removed to create an air-liquid interface, while the lower microchannel was continuously perfused with air-liquid interface medium at 60 μL/hr. In studies where physiological breathing motions were mimicked, as discussed below, cyclic strain (e.g., 10% strain at 0.2 Hz) was initiated 3 days after the creation of the air-liquid interface by applying negative pressure (e.g., about −75 kPa) to the operating channels of the device (e.g., operating channels 252).

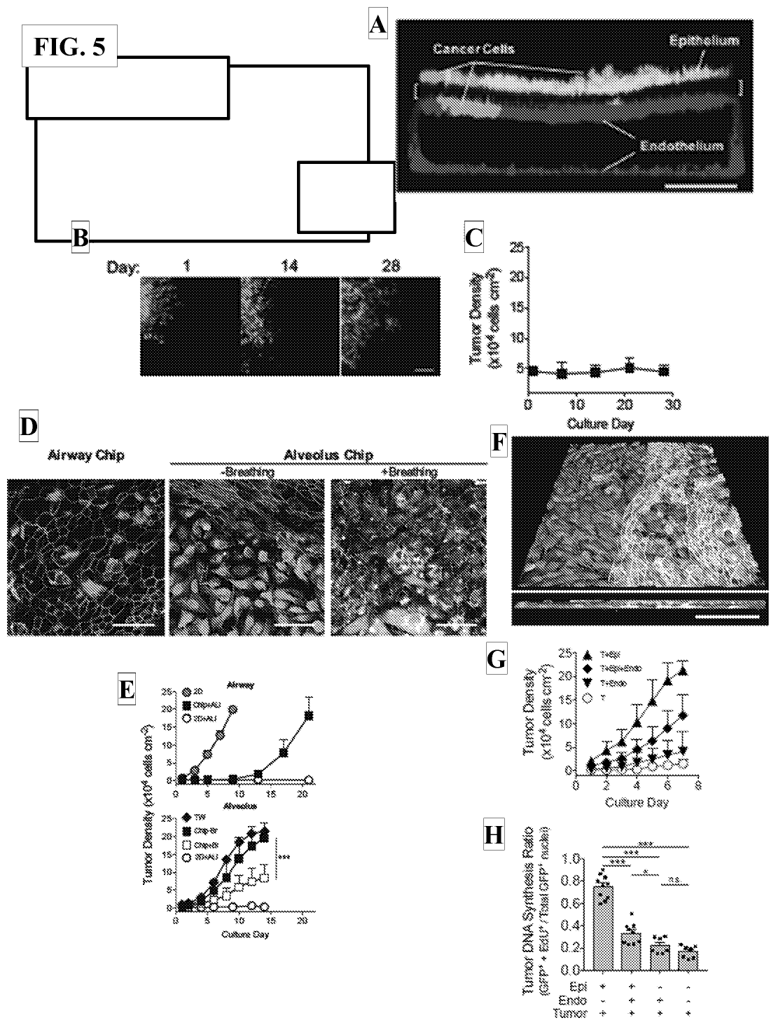

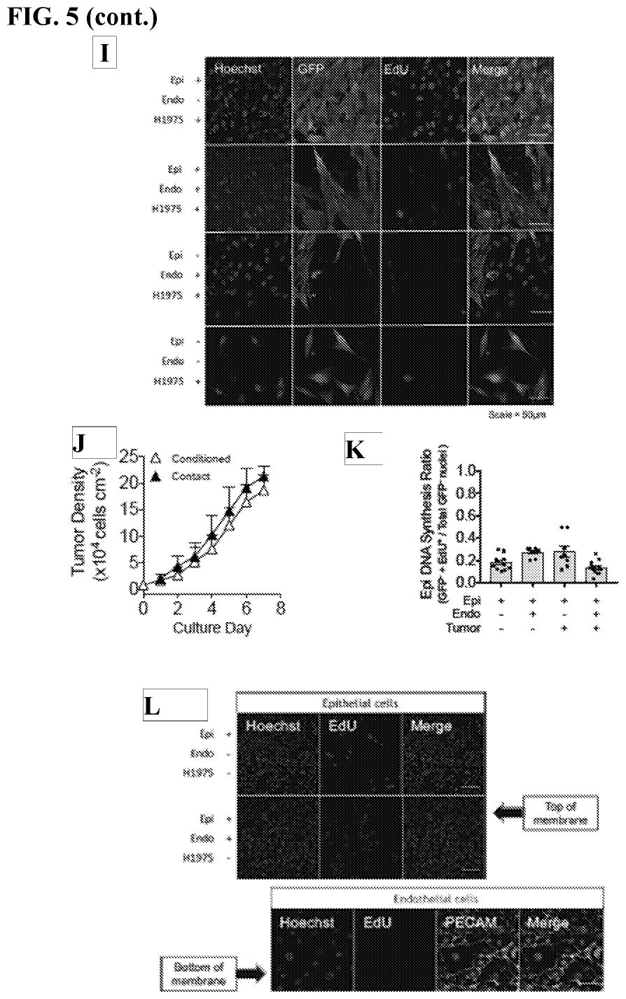

Referring to FIG. 5A, FIG. 5A illustrates the GFP-labeled cancer cells (green, anti-GFP) co-cultured with the healthy cells, such as the primary lung alveolar epithelial cells labeled with antibodies against the tight junction protein ZO-1 (white) at an air-liquid-interface in the upper microchannel of the microfluidic device. FIG. 5A shows the microfluidic device after seven days of culturing without breathing motions (bar, 200 μm). Also illustrated are the healthy cells within the lower microchannel, such as primary lung microvascular endothelial cells, which are labeled with anti-VE cadherin (red) and form a continuous monolayer that covers all four sides of the lower microchannel, creating a vascular lumen through which the ALI maintenance medium flows. As illustrated, the cancer cells grow within, above, and below the epithelial cell layer in the upper microchannel, in addition to appearing within the pores of the membrane and in the endothelial cell layer within the lower microchannel below.

The foregoing examples provide for seeding of the membrane with cancer cells prior to differentiation of healthy cells forming a healthy cell layer, such as the resulting cell layers formed of primary lung alveolar or small airway epithelial cells. In contrast to the above seeding approach, another approach for providing the cancer cells on the surface of the membrane in the upper microchannel includes injection of the cancer cells after differentiation of the healthy cell layer, such as the healthy epithelial cell layer. By way of example, 104 cells/mL of GFP-labeled NSCLC cells were loaded in a syringe with a 29 gauge needle tip, inserted through the PDMS of a microfluidic device, and injected into a differentiated layer of healthy epithelial cells on the top surface of the membrane. This was repeated until several localized areas of GFP-labeled cancer cells successfully were integrated into the epithelium.

Under the same conditions for culturing as described above for the previous examples, the cancer cell growth was inhibited, even though viable, GFP-labeled dormant tumor lesions remained present up to 4 additional weeks of culture. For example, FIG. 5B illustrates an immunofluorescence micrograph of an implanted cluster of GFP-labeled NSCLC cancer cells (green). For 1, 14, and 28 days after implantation, the cancer cells did not significantly expand in number at the site of injection. Further, FIG. 5C shows the quantification of NSCLC tumor cell densities when cultured for up to 1 month after implantation. Again, the cancer cells did not significantly expand in number at the sites of injection. Furthermore, it was not possible to inject the cancer cells directly into the pre-differentiated healthy cells, such as pre-differentiated alveolar epithelium, due to the friability of this cell monolayer.

In contrast, analysis of the growth of the NSCLC cells that were co-cultured in the microfluidic device during the differentiation process of the healthy cell layer shows that the cancer cells were isolated GFP-positive cells within the epithelial monolayer after tissue differentiation was completed (FIG. 5D). Under these conditions, the cancer cells remained quiescent for approximately 12 days of culture within the microfluidic device before they shifted into logarithmic growth and began to exhibit a doubling time of about 40 hours (FIG. 5D). Unexpectedly, although the H1975 NSCLC cell-line originates from a primary tumor and is hence expected to have mesenchymal-like features, FIG. 5D shows that the cells integrate with the healthy epithelial cells and form tight junctions—a hallmark of epithelial cells. Then, as described, this epithelial-like phenotype makes way for a mesenchymal-like phenotype, and the eventual formation of micro-nodules (discussed further below).

In contrast to the microfluidic device, when the NSCLC cells were cultured on plastic culture dishes, the NSCLC cells failed to exhibit any growth lag and proliferated even more rapidly (30 hour doubling rate) than when plated simultaneously with epithelial cells in the microfluidic device (FIG. 5D). In further contrast, the same NSCLC cells failed to exhibit any growth when cultured alone on plastic dishes. Thus, the engineered microenvironment of the microfluidic device and the process of providing the cancer cells on the membrane conveys distinct growth signals to the cancer cells, and control over the microenvironment provides for control over the growth of the cancer cells. Such control over the growth can be used for testing various agents on the cancer cells, such as various anti-cancer drugs, as further discussed below.

The foregoing demonstrates that control over the seeding of the cancer cells, including the cancer cell density and ratio to healthy cells to cancer cells, affects growth of the cancer cells within a static microfluidic environment. Whether cancer cell growth is promoted or inhibited can be controlled based on the above factors, which opens the possibility of exploring in vivo conditions that were previously not viable. Further control of the cancer cells can be provided by introducing other influences. In particular, certain microenvironments experience forces, and these forces can affect cancer cells and potentially affect cancer cell growth. Such other forces can include, for example, cyclic mechanical forces and shear forces that result from breathing motions, muscle contractions, etc.

The cancer cells described above that were co-cultured in the microfluidic device were in a predominately static environment except for culture medium flowing through the lower microfluidic channel. When the same setup was placed in a microfluidic device as described with respect to the microfluidic device 200, mechanical forces could be applied to the membrane and the cancer cells by applying cyclic pressure through the operating channels 252. The ability to expose cancer cells to such cyclic mechanical forces provides another influence on the cancer cells that inhibits their growth. For similar conditions as the static cultures described above, the cancer cells proliferated much less rapidly than the cancer cells grown in the static microfluidic environment when grown in the presence of a cyclic mechanical force (10% strain; 0.2 Hz) to mimic physiological breathing motions.

Thus, mimicking normal breathing conditions, such as the mechanical forces on the membrane and cancer cells thereon and the shearing forces of the fluid (e.g., air) moving over the membrane and the cancer cells thereon, effects various pathophysiological responses. Recapitulation of this physiologically relevant mechanical microenvironment provides for control over the cancer cell growth by suppressing the growth significantly, such as by >50% in this orthotopic model. When grown in the absence of breathing motions within a microfluidic device, the cancer cells expanded to replace large regions of the epithelium (FIG. 5D and FIG. 5F), and grew both above and below the epithelial layer (FIGS. 5A and 5F), whereas the same cancer cells remained limited to smaller localized regions of the epithelium when grown with cyclic deformation (FIG. 5E, right). Fluorescence microscopic imaging and computerized morphometric analysis of the cancer cells after 14 days of co-culture revealed that larger tumor cell clusters formed in a static microfluidic device compared to the mechanically-active microfluidic device (FIG. 5I). Breathing motions applied to the microfluidic device caused control (suppression) of the expansion into and accumulation of the cancer cells within the lower microchannel over time (FIGS. 5J and 5K).

In particular, FIG. 5I illustrates fluorescence microscopic images (top) showing GFP-labeled lung cancer cell clusters growing within the epithelial cell layer of a microfluidic device cultured for 1, 7, and 14 days in the absence (−Breathing) or in the presence (+ Breathing) and the associated cyclic mechanical deformations that mimic physiological breathing motions (bar, 100 μm). Cluster area histograms (bottom) generated with computerized image analysis confirm that larger cell clusters form in the absence of breathing motions.

With respect to FIG. 5J, illustrated are high magnification confocal fluorescence microscopic Z-stack images of GFP-labeled NSCLC cells within the breathing (e.g., microfluidic device 100, or microfluidic device 200 where breathing motions are not introduced) and non-breathing microfluidic devices (e.g., microfluidic device 200) presented in a showing that the cancer cells invade through the epithelial cell layer from the upper microchannel through the ECM-filled pores of the membrane (dashed lines) and into the endothelial layer of the lower microchannel below, and that this process is suppressed when the microfluidic device experiences breathing motions.

FIG. 5K illustrates a quantification of the invasive behavior shown in FIG. 5J where is presented as the ratio of the GFP intensity measured within the cancer cells on the lower microchannel side of the membrane to the GFP signal within tumor cells on the upper microchannel side of the membrane.

Accordingly, introducing breathing motions that result in mechanical and shear forces or influences into the microfluidic devices and to the cancer cells allows for further control of the growth of the cancer cells. The foregoing provides that control over the mechanical and shear forces applied to cancer cells influences growth of the cancer cells within a microfluidic environment. In certain microenvironment, other forces can act on the cancer cells and potentially affect cancer cell growth. Such other forces can include, for example, cyclic mechanical forces that result from muscle contractions, peristalsis contractions, and the like.

As touched on above, the healthy cells that are on the membrane with the cancer cells also can affect the growth of the cancer cells. Co-culture of cancer cells and healthy endothelial cells alone does not support cancer cell growth. In contrast, co-culture of cancer cells with healthy epithelial cells supports cancer cell growth. For example, co-culture of NSCLC cells with endothelium cells alone did not significantly support NSCLC cell growth whether measured by quantifying cell number (FIG. 5G) or incorporation of EdU into DNA (FIG. 5H and FIG. 5I). In contrast, co-culture of the NSCLC cells with healthy epithelial cells increased their growth. Moreover, co-culture of NSCLC cells with both healthy endothelial cells (density of less than 0.001) and epithelial cells further suppressed cell growth. Accordingly, the configuration of the cell layer within which the cancer cells grow has an effect on the growth, with certain healthy cell types (e.g., epithelial cells) promoting growth and certain healthy cell types (e.g., endothelial cells) inhibiting growth.

The healthy cell type that promotes or inhibits cancer cell growth can be selected based on the type of cancer. With respect to NSCLC cells as an example, the epithelial cells promoted cancer cell growth but the endothelial cells inhibited growth. Other types of cells have other interactions, such as endothelial cells promoting growth and epithelial cells inhibiting growth. Selection of the healthy cell type within which the cancer cells are integrated allows for control of the cancer cell growth for exploration of the desired cancer cell characteristics.

The microfluidic devices further allow for the testing and analysis of responses cancer cells have to one or more agents in orthotopic models that mimic conditions found in vivo. The microfluidic devices therefore open the door to new analysis that was previously not possible. Such agents can be selected from the group consisting of a small molecule, a drug or drug candidate, a chemotherapeutic, a nanoparticle, a compound, a polypeptide, a polynucleotide, or a lipid, or commensal microbes. By controlling the microenvironment within which the cancer cells are integrated, the cancer cells can be controlled to allow for exhibition of various different responses to the agents the mimic responses found in vivo and that have previously not been possible. By way of example, certain cancer cells can lay dormant despite exposure to chemotherapies or other anti-cancer drugs. By controlling the cancer cells according to the aspects of the present disclosure, microenvironments can be generated that mimic the dormant state of cancer cells found in vivo for analysis of ways to treat dormant cancer cells.

By way of example, certain cancer cells respond differently to anti-cancer drugs depending on the stage of the cancer cells. An H1975 cell line of NSCLC cells harbor an activating mutation (L858R in exon 21) and a second acquired point epidermal growth factor receptor (EGFR) mutation at T790M. While NSCLC patients with activating EGFR mutations typically have good initial responses to therapy with 1st generation reversible tyrosine-kinase inhibitors (TKIs), such as erlotinib, disease progression commonly reoccurs within 9-14 months of therapy in patients who acquire an additional EGFR mutation (e.g., T790M), which is less sensitive to this therapy. In these cases, treatment with a 3rd generation irreversible TKI that targets kinases that phosphorylate both sites, such as rociletinib, is recommended. Yet, patients with late stage NSCLC still often eventually fail to respond to therapy.

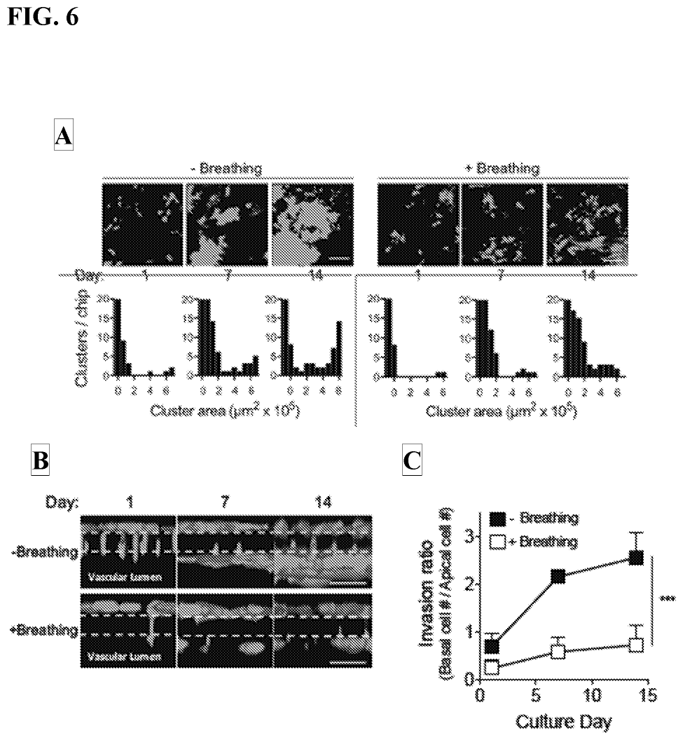

Orthotopic models based on microfluidic devices according to aspects of the present disclosure can create these same effects for analysis in vitro. For example, when H1975 NSCLC cells were cultured within the microfluidic devices, the NSCLC cells responded to 1st and 3rd generation TKIs (erlotinib and rociletinib, respectively). However, the NSCLC cells were significantly more sensitive to the inhibitory effects of rociletinib than erlotinib (half-maximal inhibition at >1000 nM vs.˜100 nM, respectively) when cultured alone under conventional static culture conditions, as shown in FIG. 6A. Specifically, FIG. 6A shows GFP-labeled cancer cell clusters growing within the epithelial cell layer of a microfluidic device, such as the microfluidic device 200, cultured for 1, 7, and 14 days in the absence (− Breathing) or presence (+ Breathing) of cyclic mechanical forces and shearing forces or influences that mimic physiological breathing motions. Thus, the microfluidic devices established that the cyclic motions, the expansion and contraction that mimic breathing, affects the performance of anti-cancer drugs.

As shown in FIG. 6B, the cancer cells were more sensitive to a TKI drug in that complete growth suppression was observed at the lower dose (100 nM; FIG. 6B, left), and the cancer cells were almost completely resistant to the inhibitory effects of the TKI drug when in the presence of physiological breathing motions (FIG. 6B, right). Further, FIG. 6C illustrates the total EGFR tyrosine phosphorylation levels (EGFR total) and levels of phosphorylation measured at tyrosines 845 (pEGFR Y845), 998 (pEGFR Y998) and 1068 (pEGFR 1068) in NSCLC cancer cells cultured for 2 days in the presence of 0, 100, 500 or 1000 nM rociletinib for 2 days in the absence (− Breathing, gray) or presence (+ Breathing, white) of cyclic mechanical strain (10%; 0.2 Hz). Phosphorylated values are normalized with respect to the corresponding total EGFR.

The effect that breathing motions within microfluidic devices have on cancer cells may be tied to tyrosine kinase activity. The decreased sensitivity of cancer cells to the TKI drug could be due to mechanical regulation of EGFR expression and signaling within the tumor cells. For NSCLC cells cultured with and without cyclic mechanical deformation (10% strain; 0.2 Hz) for 48 hours in a FlexCell culture plate, mechanical stimulation produced a significant (p<0.05) decrease in total EGFR protein levels in the NSCLC cells even before rociletinib was added, as shown in FIG. 6C. This confirms the inhibitory effects of breathing motions on cancer cell growth in FIG. 5E at the bottom and motility in FIGS. 5J and 5K. Furthermore, while treatment of cancer cells with rociletinib resulted in the inhibition of tyrosine kinase phosphorylation at EGFR sites Y845, Y998 and Y1068 in the absence of breathing motions, this inhibition also was greatly diminished in the mechanically stimulated cells, as shown in FIG. 6C). Thus, both down-regulation of EGFR and reduced suppression of phosphorylation of the EGFRs that were expressed could explain why the mechanically strained cells were resistant to growth inhibition by this 3rd generation TKI drug (FIG. 6B, right).

Further, many patients with the EGFR L858/T790M mutation who develop resistance to 3rd generation TKIs overexpress the tyrosine protein kinase c-Met, and c-MET overexpression has been implicated as a mechanism of resistance to TKI therapy in NSCLC patients. As confirmation that breathing motions decrease NSCLC cell sensitivity to this TKI, the NSCLC cells significantly increased both their expression and phosphorylation of c-Met when subjected to cyclic mechanical strain, whereas increasing rociletinib dose had no significant effect. Thus, mechanical breathing motions also may suppress NSCLC cell response to TKI therapy by altering this signaling pathway.

Moreover, it was discovered that a microfluidic device, as described herein, provided a microenvironment that allowed decreasing or eliminating the use of one or more growth factors in the cell medium used for flowing nutrients into the devices sufficient for optimal healthy cell growth, e.g., epithelial and endothelial channels. Furthermore, such lowered levels or elimination of one or more growth factors in the nutrient flow medium, contributed in part for providing a more flexible orthotropic model for identifying effects of one or more growth factors in co-cultures of healthy and cancer cells. As one example, it was discovered that EGF (Epidermal Growth Factor) supplementation may be lowered or removed from the nutrient medium for co-cultures of epithelial cells; and co-cultures of healthy epithelial cells and cancer cells. Thus, further discoveries are enabled relating to effects of endogenous EGR expression in co-cultures comprising EGFR+(Epidermal Growth Factor Receptor) expressing cancer cells; and discoveries related to endogenous EGF ligand expressing cancer cells in these devices.

Exemplary cell medium used in devices described herein, includes but is not limited to epithelial growth medium, e.g., Bronchia/Trachea Epithelial Cell Growth Medium (e.g., Sigma-Aldrich); PromoCell Airway Epithelial Cell Growth Medium is a serum-free medium (e.g., PromoCell); MEGM™ Mammary Epithelial Cell Growth Medium (e.g., Lonza); and endothelial growth medium, e.g. Endothelial Cell Growth Medium (e.g., Lonza); etc.

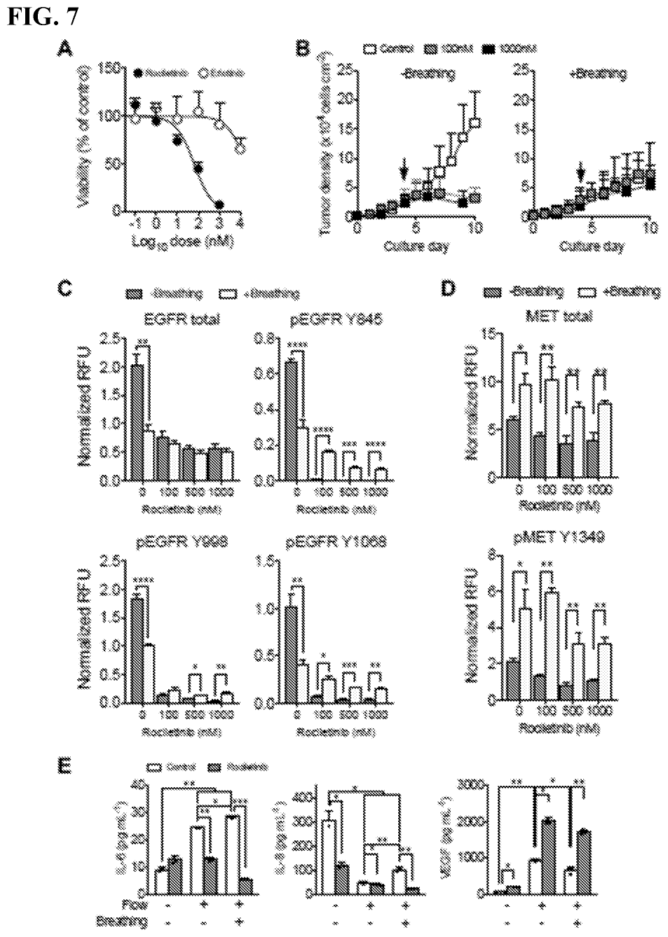

The effect that breathing motions within microfluidic chips have on cancer cells also may be tied to cytokines. The cytokines interleukin-6 (IL-6), interleukin-8 (IL-8), and VEGF may serve as clinically important prognostic indicators of cancer growth. Because the orthotropic model based on the microfluidic device features fluid flow in the upper and lower microchannels, cytokines secreted by the cancer cells can be collected and analyzed by collecting and analyzing effluent from the upper and/or lower microchannels. By way of example, analysis of the effluent from the lower microchannel revealed that IL-6 and VEGF are more abundant, whereas IL-8 was decreased, in the microfluidic device with or without breathing when compared to transwell cultures without breathing or flow (FIG. 7E). In addition, treatment of the lung tumor cells with rociletinib significantly reduced levels of IL-6 and IL-8 and increased VEGF levels in both static and breathing microfluidic devices. However, the level of suppression of the two interleukins was significantly greater in chips exposed to physiological breathing motions (FIG. 7E). For example, to compare cytokines secreted by cells, samples (400 μL) can be collected from the effluent of the microfluidic devices (e.g., from the upper and/or lower microchannel). Multiplexed cytokine measurements for VEGF, IL-6, and IL-8 can then be performed on the samples using an electrochemilluminescence immunoassay on a QuickPlex SQ 120 instrument.

Accordingly, the orthotopic models generated using the microfluidic devices according to the aspects of the present disclosure allow for modeling of complex microenvironments where that faithfully mimic microenvironment-specific growth patterns, cell secretion profiles, and clinical responses to therapy previously observed in human patients. Thus, the microfluidic devices and associated orthotopic models permit analysis of, for example, molecular level modulation of drug actions by organ microenvironments, which has not been previously possible with other in vitro cancer models or even with animal studies. This analysis can provide insight into the treatment of cancer previously not available.

Analysis of cancer cell growth and responses of cancer cells can be monitored visually through the microfluidic device. As discussed above, cancer cell growth can be monitored non-invasively using fluorescence microscopy, such as with a Zeiss TIRF/LSM 710 confocal microscopy and Hamamatsu ImagEM-1K BackThinned EMCCD camera. The number of GFP-labeled cancer cells can be estimated using fluorescence microscopy based on a standard curve generated experimentally that correlates cell density to GFP fluorescence intensity. Additionally, or in the alternative, a confocal laser-scanning microscopy system (Leica SP5 X MP) with HybriD detector or a Zeiss Axio Observer Z1 microscope with a Hamamatsu 9100-02 EMCCD can be used.

Non-invasive monitoring of cancer cell growth and response to potential or known drugs may be used for identifying drug effects, as one example, for simultaneously observing of making observations of, both healthy and cancer cells, in addition to a means for simultaneously observing effects on cancer cells in different stages of the cell cycle, i.e., proliferation. In some embodiments, observations/measurements are in real time. In some embodiments, observations/measurements are static, i.e., at a specific time or stage. In some embodiments, observations/measurements are based upon morphology, such as longer observations of co-cultures for observing solid tumor (i.e., micro-nodule) formation. In some embodiments, observations/measurements are based upon morphology in duplicate devices in parallel, such as comparing tumor formation in devices, each having one dilution of drug or growth factor from a dilution series, in turn compared to a duplicate control not receiving the drug or co-factor. In some embodiments, such micro-nodules formed within a microfluidic device are used in, but not limited to any of the assays described herein.

In addition to assays described herein assays of devices described herein include but are not limited to: cancer cell growth curves, healthy cell growth curves and comparative growth curves of seeded cancer cells and healthy cells; observations/measurements as migration (metastatic) assays, e.g., movement of cancer cells (or immune cells as described herein), such as migration from one compartment to another. One exemplary assay embodiment measures movement of cancer cells from the epithelial compartment to the endothelial compartment, in part to mimic metastasis from an organ through an endothelial cell layer into the blood stream, under comparative growth/experimental conditions described herein. In some embodiments, movement of cancer cells from the vascular compartment through the endothelium into the epithelial compartment, i.e., invasion from one compartment to another, is observed/measured under comparative growth/experimental conditions described herein. In other words, such movement of cells within one device are compared to a duplicate device under comparative growth/experimental conditions.

In some embodiments, observations include but are not limited to assays described herein, including but not limited to analysis of, effect upon or synthesis of or production of or secretion of factors, such as growth factors and interleukins, cytokines and chemokines, etc.

Use of co-cultures of healthy cells and cancer cells in devices described herein have numerous advantages over growing cancer cells alone. These advantages include, but not limited to, overcoming limitations of growing cancer cells in a dish, in part because cancer cells grow at a much faster rate than healthy cells, thus overgrowing dishes and using nutrients at a faster rate than healthy cells, i.e., creating in part a rapidly developed acidic type-nutrient deprived microenvironment, thus under these type of growth conditions, microenvironmental effects related to the development of cancer cells and microtumors, are not physiological relevant. As one example, when cell medium is collected from healthy cells growing in culture then added to cancer cells seeded at low density on chips, these cancer cells don't grow. In contrast, when healthy epithelial cells are co-cultured with cancer cells, the cancer cells grow. Thus, healthy cells, i.e., a healthy cell microenvironment, create a microenvironment stimulating the cancer cells to grow. “Low density” refers to a density typically in use for drug assays allowing room to grow/expand in culture. Surprisingly, while co-culture of healthy cells and cancer cells in plates demonstrates patches of cancer cell growth, cancer cell growth is observed evenly and reproducibly throughout the device channels. Moreover, healthy endothelial cells also had a surprising effect, such that epithelial cells stimulated cancer cell growth however healthy endothelial cells suppressed this growth. In some embodiments, the presence of endothelial cells led to EDU uptake to be reduced preferentially in cancer cells. In some embodiments, changes in cytokine expression were observed. In some embodiments, endothelial cells modulated VGEF and VGEF-R expression. These results indicate that the microfluidic device of the present invention is capable of recapitulating aspects of cancer biology relating to interaction with the endothelium. Similarly, experimental results (e.g. FIG. 7) indicate that the microfluidic device of the present invention is capable of recapitulating aspects of cancer biology relating to mechanical forces. As such, the microfluidic devices of the present invention lend themselves to a greater extent than prior in vitro technologies to the study of cancer biology. Moreover, through the further introduction of an agent, the present invention enables improved evaluation of agent efficacy, toxicity and/or mechanism of action. In some embodiments, an agent was introduced to the microfluidic device (e.g., rociletinib), and the effect of such agent was compared with and without endothelial cells present and/or with and without the presence of mechanical stretch (e.g., FIG. 7E).

In some embodiments, devices comprising co-cultures of healthy cells and cancer cells as described herein are used for identifying cancer cell effects upon healthy cells in co-culture. Examples include but are not limited to identifying synergistic effect on cytokine expression, gene expression, effects upon immune cells added to these co-cultures, etc.

In some embodiments, devices described herein are used for drug target discovery.

Another assay for evaluating cell growth/maturation and/or changes in morphology or cell type, includes but is not limited to observing and measuring Epithelial-mesenchymal transition (EMT) and its reverse, mesenchymal-epithelial transition (MET). EMT and MET refer to developmental programs which were shown to have roles in promoting metastasis and invasion, as well as contribute to drug resistance in cancer cells. As one example, ATCC has employed CRISPR/Cas9 gene editing to develop a reporter line designed to enable the real-time monitoring of the changing status of these cells from epithelial to mesenchymal. As such, an A-549 VIM RFP (ATCC® CCL-185EMT™) human epithelial lung cancer cell was engineered for epithelial to mesenchymal transition (EMT) for use in anti-EMT drug screening, metastatic non-small cell lung cancer drug screening, vimentin intermediate filament dynamics, etc. Thus in some embodiments, a cancer cell for use herein is a lung cancer cell A-549 VIM RFP (ATCC® CCL-185EMT™). As an example, lung cancer cell A-549 VIM RFP (ATCC® CCL-185EMT™) in culture undergoing EMT expressing vimentin, aSMA, n-cadherin may continue to make tight junctions; however, at least by 2 weeks in culture may begin to form micro-nodules. In some embodiments, micro-nodule formation may represent an additional EMT moving the cells further towards a mesenchymal-like morphology

In some embodiments, any of the cancer cell types described herein may be observed and/or evaluated for spontaneous EMT or MET transformations. In some embodiments, any of the cancer cell types described herein may be engineered for observing indications of or transitions of EMT or MET transformations. As one example, increasing expression of epithelial-mesenchymal transition (EMT)-related proteins, including but not limited to E-cadherin, N-cadherin, aSMA and vimentin, may be compared between control breast cancer cell lines and cells that are known or suspected of EMT. In reverse, control breast cancer cell lines and cells that are known or suspected of MET may be evaluated for decreasing expression of MET related proteins, or gene expression, including but not limited to E-cadherin, N-cadherin, aSMA and vimentin. Additional assays for EMT or MET may include changed in capability of forming tight junctions, such that more epithelial-like cells transitioning to mesenchymal cells may decrease in capability to form tight junctions while more mesenchymal-like cells may increase in capability to form tight junctions. In some embodiments, such spontaneous transformations may be in response to experimental microenvironments. In some embodiments, such spontaneous transformations may be in response to drug treatments.

Immunostaining studies also can be used to analyze cell growth. For example, microfluidic devices can be provided with antibodies directed against GFP, ZO1, and VE-cadherin to visualize cancer cells, epithelial tight junctions, and endothelial cell-cell adhesions, respectively. Further, one or more cells, such as healthy cells and cancer cells, can be removed from the microfluidic devices for analysis.

Further, as described above, effluent from the microfluidic devices can be collected from the various microchannels. The effluent can then undergo various testing to determine the responses of the cancer cells to various chemotherapies and anti-cancer drugs that are perfused through the microfluidic devices. By being able to control the cancer cells, such as causing the cancer cells to remain dormant, chemotherapy and anti-cancer drug therapies can be tested to determine new cancer therapies that destroy dormant cancer cells (e.g., persister cells). The microfluidic devices also can be used to analyze mechanisms by which anti-cancer drugs do and do not inhibit growth or invasion of the cancer cells through optically monitoring the responses of the cancer cells to the anti-cancer drugs.

By way of specific examples, using the microfluidic devices configured as described above, effluents can be collected for multi-omics analysis of epithelial and vascular channels independently, recruitment of circulating immune cells can be analyzed, living human cells with normal organ-specific microbiome can be co-cultured, and multiple microfluidic devices can be linked in a more physiologically relevant way (i.e., fluidically via their vascular channels).

In one or more embodiments, the analysis can include identifying one or more cancer cells within the microfluidic devices and removing the cancer cells from the microfluidic devices for further analysis. Such cancer cells identified can be cancer cells that are dormant and resistant to anti-cancer drugs perfused through the microfluidic device, as an example. These cancer cells can be excised from the microfluidic devices for additional testing to determine the cellular mechanisms that allow for the cancer cells to remain dormant and resistant.

Further, although the present disclosure focuses primarily on NSCLC cells, the cancer cells can be any type of cancer cells, such as breast cancer cells, colorectal cancer cells, pancreatic cancer cells, kidney cancer cells, prostate cancer cells, urothelial cancer cells, oesophageal cancer cells, head and neck cancer cells, hepatocellular cancer cells, mesothelioma cells, Kaposi's sarcoma cells, ovarian cancer cells, soft tissue sarcoma cells, glioma, melanoma cells, small-cell and non-small-cell lung cancer cells, endometrial cancer cells, basal cell carcinoma cells, transitional cell carcinoma of the urothelial tract, cervical cancer cells, endometrial cancer cells, gastric cancer cells, bladder cancer cells, uterine sarcoma cells, multiple myeloma cells, soft tissue and bone sarcoma cells, cholangiocarcinoma cells, or a cancer cells disseminated therefrom.

In one embodiment, a system having at least two organ on a chip devices can be coupled through at least one or more fluid sources and/or pressure sources. It is contemplated that at least 3, at least 4, at least 5, at least 6, or at least 7 or more organ on a chip devices can be coupled through at least one or more fluid sources and/or pressure sources. In one example, the fluid from a fluid source the at least two devices can be connected in parallel with respect to the fluid source. In one embodiment, the at least two devices can be connected in serial fashion with respect to the fluid source. In yet another embodiment, when at least three or more devices are coupled, they can be connected in both parallel and in serial fashion with respect to the fluid source.

With multiple devices operating, it is possible to monitor, using sensor data, how the cells in the fluid or membrane behave after the fluid has been passed through another controlled environment. This system thus allows multiple independent “stages” to be set up, where cell behavior in each stage may be monitored under simulated physiological conditions and controlled using the devices. One or more devices are connected serially may provide use in studying chemical communication between cells. For example, one cell type may secrete protein A in response to being exposed to a particular fluid, whereby the fluid, containing the secreted protein A, exits one device and then is exposed to another cell type specifically patterned in another device, whereby the interaction of the fluid with protein A with the other cells in the other device can be monitored (e.g. paracrine signaling). For the parallel configuration, one or more devices connected in parallel may be advantageous in increasing the efficiency of analyzing cell behavior across multiple devices at once instead of analyzing the cell behavior through individual devices separately.

In one embodiment, a system in which at least two devices are connected can be used to study a metastatic cancer model. In one embodiment, the upstream microfluidic device comprises a microenvironment seeded with cancer cells that match said microenvironment (for example, a lung on a chip seeded with NSCLC cells), and the downstream microenvironment being a different microenvironment from the upstream microenvironment (for example, the upstream microenvironment is a lung on a chip, and the downstream microenvironment is a liver on a chip). This system allows for the analysis of cancer cell properties as it travels from the upstream microenvironment to the downstream microenvironment.

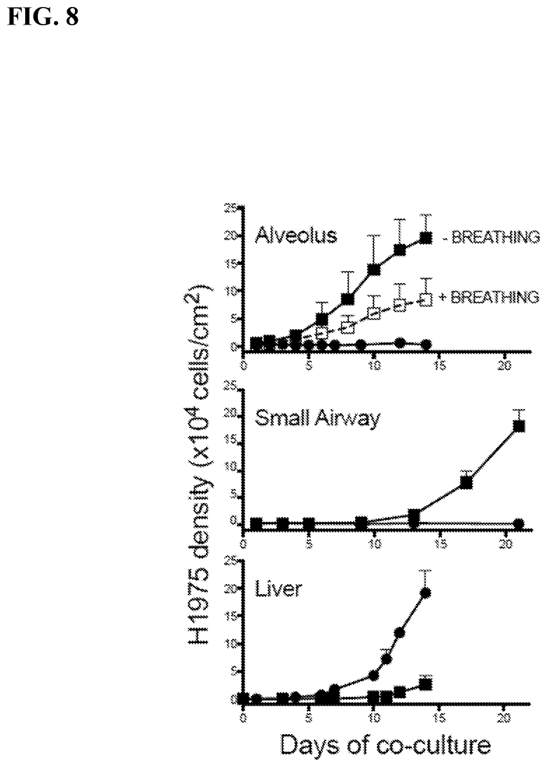

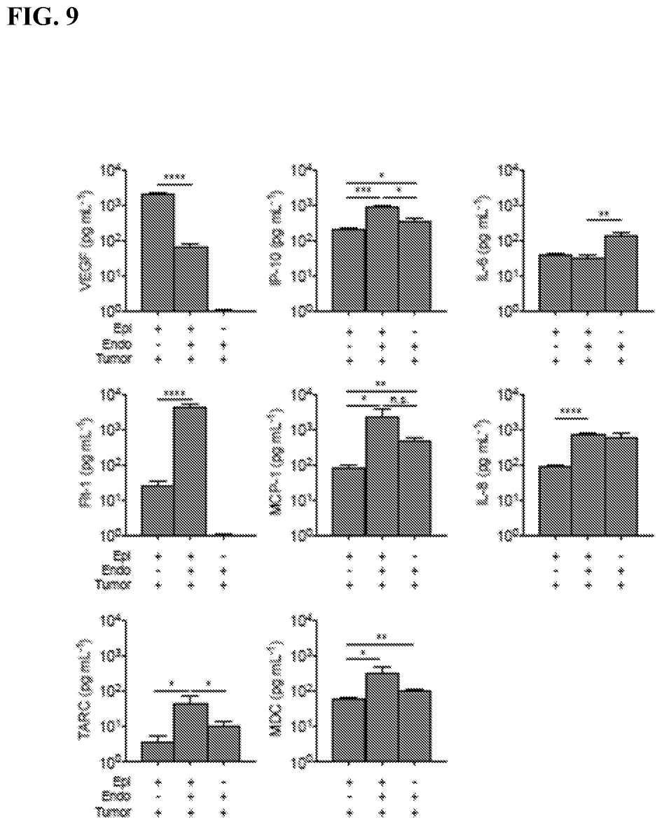

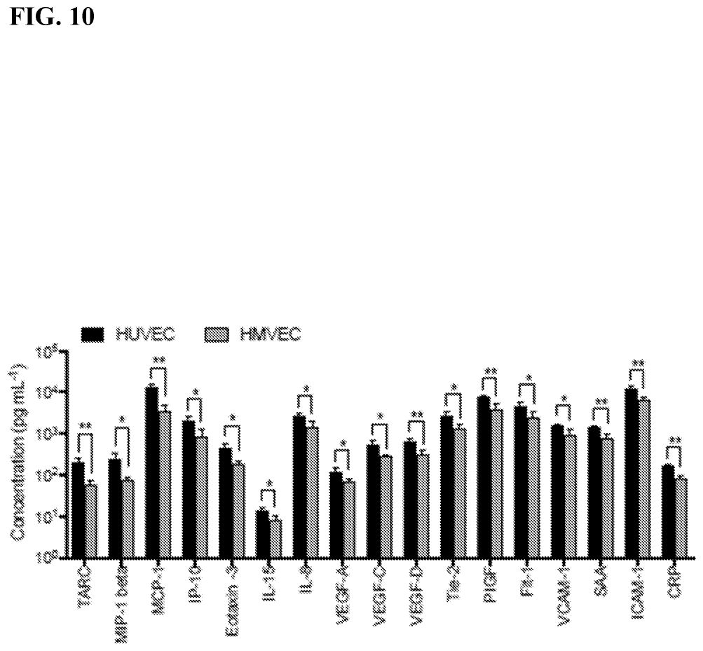

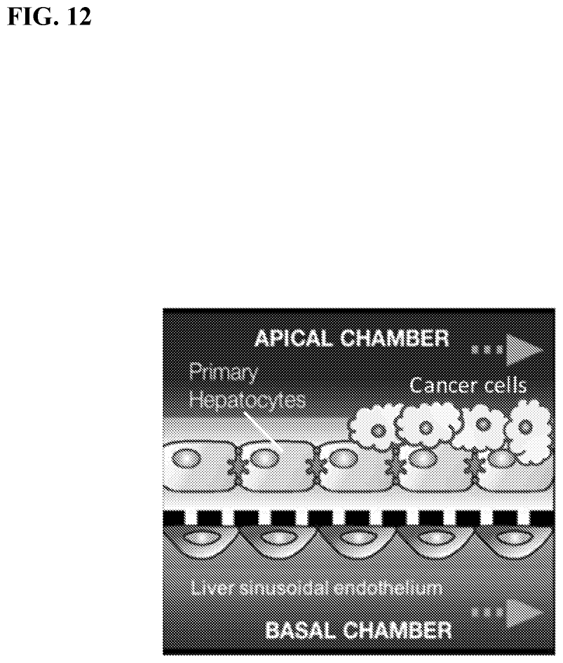

In one example, the present invention can be used to explore aspects that influence cancer cell growth and behavior in microenvironments that differ from the cancer cells' origin. Such microenvironments are referred to as metastatic models where the cancer cells are in an environment different from their origin, such as liver cancer cells within a lung environment, lung cancer cells within a liver environment, and the like. In one or more embodiments, a model of a metastatic liver lesion is provided. However, the model can be used for any type of metastatic model. The provided model includes implanted NSCLC adenocarcinoma cell line (H1975) cells in a microfluidic device configured as a human liver. The microfluidic device includes primary human hepatocytes in an apical microchannel and liver sinusoidal endothelium lining a basal microchannel. The microfluidic device further includes an ECM coated porous membrane as described herein separating the apical and basal microchannels. For example, the porous membrane is lined with the primary human hepatocytes on one side and the liver sinusoidal endothelium on the opposing side (FIG. 12). NSCLC cells are seeded on the primary human hepatocytes. The growth of the NSCLC cells was significantly suppressed on the microfluidic device compared to NSCLC cells grown in a 2D co-culture with the liver derived cells in the same medium (FIG. 8, bottom bar graph). Thus, the NSCLC cells exhibit differential behavior when present in various microenvironments. The NSCLC cells exhibit a more rapid growth within a lung microenvironment modeled using a microfluidic device compared to a liver microenvironment modeled using a microfluidic device. In some embodiments, brain cells suppressed growth of nonbrain derived cancer cells. Thus, healthy cells in non-orthotopic locations, i.e. nonprimary organ sites; other-organ healthy cells, suppressed cancer growth in microfluidic devices described herein.

This data recapitulates clinical observations that show differential intrapulmonary growth in patients with the adenocarcinoma form of NSCLC, as well as the dormancy that many types of cancer exhibit for years at metastatic sites. Thus, these findings provide proof-of-concept for using human microfluidic devices to mimic human cancer pathophysiology in vivo, and demonstrate the utility of these microfluidic culture devices as representative surrogates that mimic relevant organ-specific microenvironments for primary and metastatic cancer models.

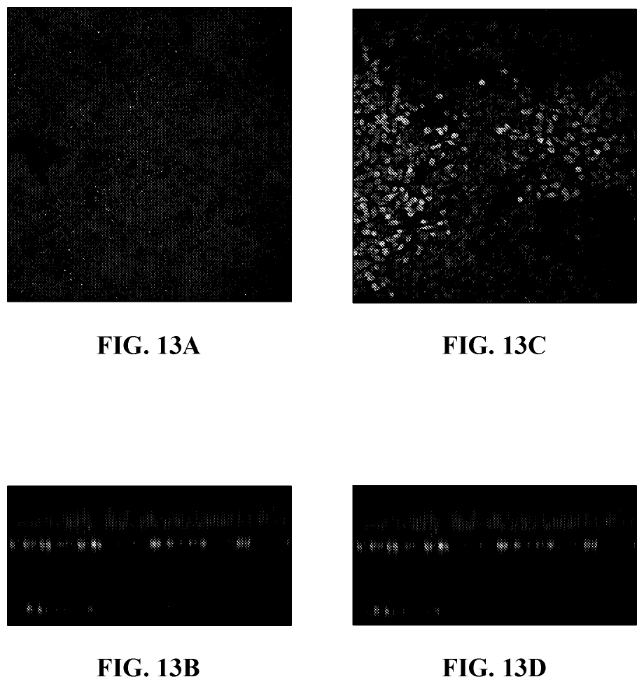

Referring to FIGS. 13A through 13D, these figures illustrate the co-culture of healthy and colon cancer cells. Specifically, normal organoid-derived human colonic epithelial cells were co-cultured with human HT29 colon cancer cells (visualized by GFP-labeling) in the upper (apical) channel. Human intestinal microvascular endothelial cells were cultured on the lower (basal) surface of the membrane. Fluorescent microscopy revealed that GFP-labeled HT29 colon cancer cells migrated to bottom channel following incubation. Chromosomal DNA is visualized via DAPI staining.

More specifically, the devices disclosed herein were used to co-culture cancer cells, e.g., HT29 colon cancer cells, and healthy epithelial cells, e.g., normal, primary human colon organoid epithelial cells, under conditions that promote the formation of tight junction between the cancer and healthy cells.

Chips were fabricated from PDMS and assembled as described herein. Chips were activated by oxygen plasma treatment for 1.5 min followed by incubation with APTMS (2% vol/vol in ethyl alcohol) for 30 min at RT, washing in ethyl alcohol, and incubating the devices at 80° C. overnight. Type I collagen (200 μg ml-1) and Matrigel (1% in PBS) were then introduced into the channels, and incubated in a humidified 37° C. incubator for 2 h before washing with PBS.

HIMEC (Human Intestinal Microvascular Endothelial Cells) were seeded in the bottom channel of the device at a seeding density of 120,000/cm2. Following seeding, the chip was inverted and endothelial cells were incubated for 2 h. GFP-labeled HT29 colon cancer cells were seeded in the top channel of the device at a seeding density of 6,000/cm2. HT29 cells were incubated for 4 h. Primary human colon organoid epithelial cells (derived from human colon resections) were processed for seeding according to the protocol found in Kasendra, M., et al. Scientific Reports; 8, Article number: 2871 (2018), which is incorporated herein by reference in its entirety. Briefly, organoids were isolated from matrigel, enzymatically fragmented with TrypLE supplemented with 10 μM Y-27632, and seeded at 700,000/cm2. The final ratio of HT29 cells to organiod cells was 1 to 116. Cells were incubated overnight.

Following cell seeding, the chip was washed with expansion medium (i.e., stem cell medium) and attached to the ZOE. The apical channel was exposed to Hank's balanced salt solution (HBSS) with the antibiotic primocin (antibiotic). The bottom channel was exposed to expansion medium containing the EGMTM-2 MV Microvascular Endothelial Cell Growth Medium-2 SingleQuots™ Supplements and Growth Factors (Lonza, Catalog #: CC-4147; Allendale, N.J.) to support the endothelial cells at 60 ul/h flow rate.

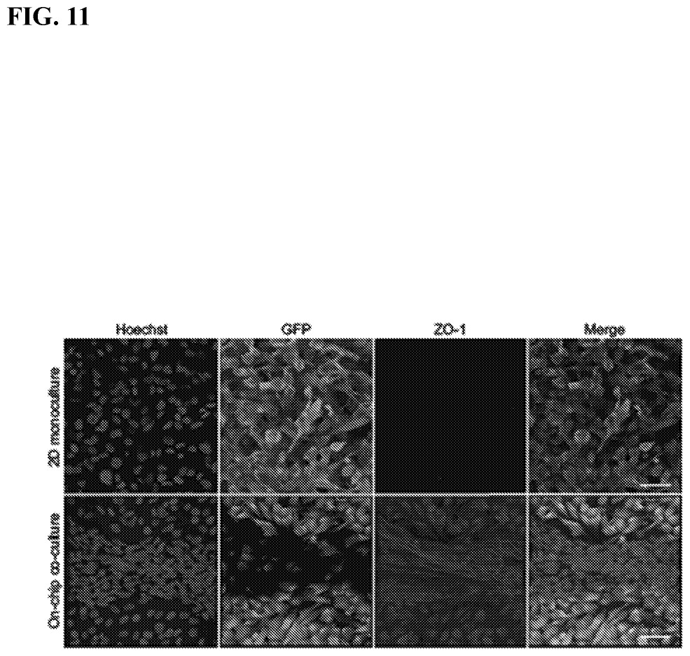

GFP-labeled HT29 cancer cell growth and migration was monitored using epi-fluorescent microscopy. 20 days post-seeding, cells in both channels were fixed using standard techniques, e.g., with 4% PFA, and stained with Hoechst to visualize the nuclei and anti-GFP antibody to detect the GFP-labeled cancer cells. Standard confocal microscopy was used to analyze fixed cells (FIG. 11).

As shown in FIGS. 13A-13D, GFP-labeled HT29 cancer cells were observed to migrate from the upper channel comprising the cancer cell/healthy cell co-culture to the lower channel comprising human intestinal microvascular endothelial cells.

Although the present disclosure focuses on healthy cells and cancer cells, such that the healthy cells are not cancerous and have a normal cell microbiome, in some aspects the concepts of the present disclosure can be applied to various other cellular investigations related to any type of disease of the cell. For example, healthy cell as used throughout can be any type of cell that does not suffer from a certain disease, and cancer cell as used throughout can be any type of cell that suffers from a disease being investigated, not necessarily cancer.

For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.” Additionally, the singular terms “a,” “an,” and “the” include plural referents unless context clearly indicates otherwise.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

Some embodiments of the technology described herein can be defined according to any of the following numbered paragraphs:

1. A method comprising:
providing a first microfluidic device having a body, the body including a first microchannel separated from a second microchannel by an at least partially porous membrane, the membrane having a first side facing the first microchannel and a second side facing the second microchannel;
seeding the first side of the membrane with healthy cells and cancer cells, forming a tissue layer; and
culturing the healthy cells and the cancer cells within the first microfluidic device by flowing fluid through one or more of the first and second microchannels, wherein the density of the cancer cells adhered to the first side of the membrane is in a range such that the culturing of the healthy cells and the cancer cells causes the cancer cells to integrate into the tissue layer formed of healthy cells.
2. The method of paragraph 1, wherein at least some of said cancer cells and at least some of said healthy cells correspond to the same organ.
3. The method of paragraph 1, wherein at least some of said cancer cells and at least some of said healthy cells correspond to the different organs.
4. The method of any of the preceding paragraphs, wherein said cancer cells are seeded prior to said healthy cells.
5. The method of any of the preceding paragraphs, wherein the culturing of the healthy cells and the cancer cells causes the cancer cells to form tight junctions with said healthy cells.
6. The method of any of the preceding paragraphs, wherein the healthy cells are differentiated and said cancer cells grow more slowly in the presence of said differentiated healthy cells than in the presence of undifferentiated healthy cells.
7. The method of any of the preceding paragraphs, wherein immune cells are included within the tissue layer in the first microchannel and/or a tissue layer in the second microchannel.
8. The method of any of the preceding paragraphs, wherein the culturing comprises flowing a culturing medium through the second microchannel while air is present in the first microchannel.
9. The method of any of the preceding paragraphs, wherein the culturing comprises flowing a culturing medium through the first and second microchannels.
10. The method of any of the preceding paragraphs, wherein the healthy cells seeded in the first channel comprise epithelial cells.
11. The method of any of the preceding paragraphs, further comprising seeding second healthy cells in at least a portion of the second microchannel, the second side of the membrane, or a combination thereof.
12. The method of paragraph 11, wherein said second healthy cells comprise endothelial cells.
13. The method of any of the preceding paragraphs, wherein a ratio of the healthy cells to the cancer cells adhered on the first side of the membrane is between about 25:1 and about 500:1.
14. The method of paragraph 13, wherein the ratio of the healthy cells to the cancer cells adhered on the first side of the membrane is about 100:1.
15. The method of any of the preceding paragraphs, wherein a density of the cancer cells adhered to the first side of the membrane is between about 100 to about 10,000 cells/cm2.
16. The method of paragraph 15, wherein the density of the cancer cells adhered to the first side of the membrane is about 3200 cells/cm2.
17. The method of any of the preceding paragraphs, wherein the membrane is coated with at least one attachment molecule that supports adhesion of the healthy cells, the cancer cells, or a combination thereof.
18. The method of any of the preceding paragraphs, further comprising applying a fluidic shear force across the membrane within the first microchannel, second channel, or a combination thereof.
19. The method of any of the preceding paragraphs, further comprising applying a mechanical force to the healthy cells, cancer cells, or a combination thereof.
20. The method of paragraph 19, wherein the said applying of a mechanical force comprises applying a mechanical force to the membrane.
21. The method of paragraph 18, wherein the fluidic shear force controls growth of the cancer cells by inhibiting growth as compared to absence of the fluidic shear force.
22. The method of paragraph 18, wherein the fluidic shear force, the mechanical force, or combination thereof controls growth of the cancer cells as compared to absence of the said shear force, mechanical force, or the combination thereof.
23. The method of paragraph 18, wherein the fluidic shear force mimics a shear force of air within a lung during breathing motions.
24. The method of paragraph 18, wherein the fluidic shear force mimics a shear force of blood flowing through a vessel.
25. The method of paragraph 19, wherein the mechanical force mimics the expansion and contraction of a lung during breathing motions.
26. The method of paragraph 19, wherein the mechanical force mimics the motion of at least one portion of the intestine during peristaltic motions.
27. The method of paragraph 18, further comprising:
applying one or more agents to the healthy cells, the cancer cells, or a combination thereof; and analyzing the healthy cells, the cancer cells, or a combination thereof to determine effects of the one or more agents.
28. The method of paragraph 27, wherein the one or more agents are selected from the group consisting of a small molecule, a drug or drug candidate, a chemotherapeutic, a nanoparticle, a compound, a polypeptide, a polynucleotide, a lipid, immunomodulator, and microbes.
29. The method of paragraph 28, wherein the one or more agents are one or more anti-cancer drugs, and the analyzing is of effects the one or more anti-cancer drugs have on the cancer cells.
30. The method of paragraph 27, wherein the analyzing comprises detecting the molecular level modulation of drug action.
31. The method of paragraph 29, wherein the one or more anti-cancer drugs are one or more tyrosine-kinase inhibitors.
32. The method of any of the preceding paragraphs, further comprising:
applying the one or more agents to the healthy cells, the cancer cells, or a combination thereof prior to, during, and/or after the application of a fluidic shear force, mechanical force, or a combination thereof; and
analyzing the healthy cells, the cancer cells, or a combination thereof to determine effects of the one or more agents.
33. The method of paragraph 32, further comprising comparing the effects of the one or more agents applied with and without the application of the fluidic shear force, the mechanical force, or a combination thereof.
34. The method of any of the preceding paragraphs, further comprising evaluating the migration of cancer cells between said first and second microchannels.
35. The method of any of the preceding paragraphs, wherein the healthy cells are primary cells.
36. The method of paragraph 35, wherein the primary cells comprise more than one primary cell type.
37. The method of any of the preceding paragraphs, wherein the healthy cells are mammalian primary cells.
38. The method of any of the preceding paragraphs, wherein the healthy cells are human primary cells.
39. The method of any of the preceding paragraphs, wherein the healthy cells are primary epithelial cells.
40. The method of any of the preceding paragraphs, wherein the healthy cells are primary endothelial cells.
41. The method of any of the preceding paragraphs, wherein the healthy cells are primary stromal cells.
42. The method of any of the preceding paragraphs, wherein the healthy cells are primary lung cells.
43. The method of any of the preceding paragraphs, wherein the healthy cells are lung alveolar or airway epithelial cells.
44. The method of any of the preceding paragraphs, wherein the healthy cells are liver hepatocyte cells.
45. The method of any of the preceding paragraphs, wherein the healthy cells are intestinal epithelial cells.
46. The method of any of the preceding paragraphs, wherein the healthy cells are sinusoidal endothelial cells.
47. The method of any of the preceding paragraphs, wherein the cancer cells are primary cancer cells.
48. The method of paragraph 47, wherein the primary cancer cells are human primary cancer cells.
49. The method of any of the preceding paragraphs, wherein the cancer cells are a cancer cell line.
50. The method of paragraph 49, wherein the cancer cell line is established from human tissue.
51. The method of any of the preceding paragraphs, wherein the cancer cells are lung cancer cells.
52. The method of paragraph 51, wherein the lung cancer cells are non-small cell lung cancer cells.
53. The method of paragraph 52, wherein the non-small cell lung cancer cells are non-small cell lung cancer adenocarcinoma cells.
54. The method of any of the preceding paragraphs, wherein the cancer cells are metastatic cancer cells.
55. The method of any of the preceding paragraphs, wherein the healthy cells and the cancer cells are derived from the same tissue type.
56. The method of any of the preceding paragraphs, wherein the healthy cells and the cancer cells are not derived from the same tissue type.
57. The method of any of the preceding paragraphs, further comprising contacting the healthy cells, the cancer cells, or a combination thereof with at least one agent.
58. The method of paragraph 57, further comprising measuring a response of the healthy cells, the cancer cells, or a combination thereof to the at least one agent.
59. The method of paragraph 58, further comprising extracting the cancer cells from the first microfluidic device prior to measuring the response.
60. The method of paragraph 57, further comprising measuring products of the cancer cells or healthy cells from an effluent of the first microfluidic device.
61. The method of paragraph 57, further comprising assessing viability of the cancer cells after the contacting.
62. The method of any of the preceding paragraphs, wherein the cancer cells are breast cancer cells, colorectal cancer cells, pancreatic cancer cells, kidney cancer cells, prostate cancer cells, urothelial cancer cells, oesophageal cancer cells, head and neck cancer cells, hepatocellular cancer cells, mesothelioma cells, Kaposi's sarcoma cells, ovarian cancer cells, soft tissue sarcoma cells, glioma, melanoma cells, small-cell and non-small-cell lung cancer cells, endometrial cancer cells, basal cell carcinoma cells, transitional cell carcinoma of the urothelial tract, cervical cancer cells, endometrial cancer cells, gastric cancer cells, bladder cancer cells, uterine sarcoma cells, multiple myeloma cells, soft tissue and bone sarcoma cells, cholangiocarcinoma cells, or a cancer cells disseminated therefrom.
63. The method of any of the preceding paragraphs, further comprising imaging the cancer cells within the first microfluidic device.
64. The method of paragraph 63, further comprising:
modifying the cancer cells to express a fluorescent protein, wherein the fluorescent protein promotes imaging of the cancer cells.
65. The method of any of the preceding paragraphs, further comprising monitoring growth of the cancer cells.
66. The method of any of the preceding paragraphs, further providing a second microfluidic device in fluid connection downstream of the first microfluidic device.
67. The method of paragraph 66, wherein the type of healthy cells comprised in the first microfluidic device and the second microfluidic device are different.
68. The method of paragraph 66, wherein the flowing medium flows through the first microfluidic device to the second microfluidic device.
69. The method of paragraph 66, wherein the cancer cells seeded in the first microfluidic device travel to the second microfluidic device.
70. The method of paragraph 66, wherein the cancer cells seeded in the first microfluidic device integrate into the tissue layer formed of differentiated healthy cells of the second microfluidic device.
71. The method of paragraph 66, wherein the cancer cells and the healthy cells seeded in the first microfluidic device are derived from the same tissue type.
72. The method of paragraph 66, wherein the cancer cells and the healthy cells seeded in the first microfluidic device are derived from a different tissue type.
73. The method of paragraph 66, wherein the cancer cells seeded in the first microfluidic device and the healthy cells seeded in the second microfluidic device are derived from a different tissue type.
74. The method of a paragraph 66, wherein the healthy cells and the cancer cells seeded in the first microfluidic device are derived from the lung; and the healthy cells seeded in the second microfluidic device are derived from the liver.
75. A method comprising:
a) providing i) cancer cells having one or more mesenchymal-like features, ii) healthy epithelial cells, and a fluidic device comprising a membrane; and
b) co-culturing said cancer cells and said healthy epithelial cells on a first surface of the membrane under conditions such that at least a portion of said cancer cells form tight junctions with said healthy epithelial cells.
76. The method of paragraph 75, wherein the cancer cells are provided on the membrane at a density range of about 100 to about 10,000 cells/cm².
77. The method of paragraph 76, wherein the density range controls the growth of the cancer cells compared to outside the density range.
78. The method of paragraph 76, wherein the cancer cells are provided on the membrane at a density about 3200 cells/cm².
79. The method of any of the preceding paragraphs, wherein the cancer cells are provided on the membrane at a ratio of the healthy cells to cancer cells of about 25:1 and about 500:1.
80. The method of paragraph 79, wherein the ratio controls the growth of the cancer cells compared to outside of the ratio.
81. The method of any of the preceding paragraphs, further comprising differentiating said healthy epithelial cells into a differentiated layer, wherein the cancer cells are seeded on said membrane prior to or after differentiating of the healthy cells into the differentiated layer.
82. The method of paragraph 81, wherein seeding the cancer cells prior to or after differentiating of the healthy cells into the differentiated layer controls the growth of the cancer cells.
83. The method of paragraph 76, wherein the cancer cells are provided after differentiating of the healthy cells into the differentiated layer.
84. The method of paragraph 83, wherein seeding with the cancer cells after differentiating of the healthy cells into the differentiated layer controls the growth of the cancer cells to inhibit cancer cell growth.
85. The method of any of the preceding paragraphs, further comprising continuing to co-culture until said tumor cells progress to form nodules.
86. The method of any of the preceding paragraphs, further comprising contacting the healthy cells, the cancer cells, or a combination thereof with at least one agent.
87. The method of Paragraph 86, wherein said agent kills at least a portion of said cancer cells.
88. The method of Paragraph 85, further comprising contacting the co-culture with an agent that inhibits formation of said nodules.
89. The method of any of the preceding paragraphs, wherein said one or more mesenchymal-like features are selected from the group consisting of expression of vimentin, expression of aSMA, and expression of n-cadherin.
90. The method of Paragraph 85, wherein at least a portion of said cancer cells transmigrate said membrane.
91. The method of any of the preceding paragraphs, wherein said fluidic device is a transwell.
92. The method of any of the preceding paragraphs, wherein said fluidic device is a microfluidic device.
93. The method of any of the preceding paragraphs, wherein at least a portion of said cancer cells in step b) undergo a mesenchymal-epithelial transition.
94. A fluidic device comprising:
a membrane; and
a first cell layer formed on a first side of the membrane, the first cell layer comprising first healthy cells and cancer cells, the cancer cells being integrated into the first cell layer and having tight junctions with said healthy cells.
95. The device of paragraph 94, wherein the first healthy cells are epithelial cells.
96. The device of any of the preceding paragraphs, wherein the cancer cells are adhered to the membrane at a cell density of about 100 to about 10,000 cells/cm2.
97. The device of any of the preceding paragraphs, wherein the cell density is about 3200 cells/cm2.
98. The device of any of the preceding paragraphs, wherein a ratio of the first healthy cells to the cancer cells adhered on the first side of the membrane is between about 25:1 and about 500:1.
99. The device of paragraph 98, wherein the ratio is about 100:1.
100. The device of any of the preceding paragraphs, further comprising a second cell layer formed at least on some portion of the second side of the membrane, the second cell layer comprising second healthy cells.
101. The device of paragraph 100, wherein said second cell layer comprises endothelial cells.
102. The device of any of the preceding paragraphs, further adapted to permit mechanical strain.
103. The device of any of the preceding paragraphs, wherein at least some of said cancer cells and at least some of said healthy cells correspond to the same organ.
104. The device of any of the preceding paragraphs, wherein at least some of said cancer cells and at least some of said healthy cells correspond to the different organs.
105. The device of any of the preceding paragraphs, wherein said fluidic device is a transwell.
106. The device of any of the preceding paragraphs, wherein said fluidic device is a microfluidic device
107. The device of Paragraph 106, wherein said microfluidic device comprises a first microchannel and a second microchannel, with the membrane separating the first microchannel from the second microchannel, the membrane having a first side facing the first microchannel and a second side facing the second microchannel.
108. A method comprising:
a) providing i) cancer cells having one or more mesenchymal-like features, ii) healthy epithelial cells, and a fluidic device comprising a membrane;
b) co-culturing said cancer cells and said healthy epithelial cells on a first surface of the membrane under conditions such that at least a portion of said cancer cells form tight junctions with said healthy epithelial cells; and
c) continuing to co-culture until at least a portion of said cancer cells lose said tight junctions with said healthy epithelial cells.
109. The method of Paragraph 108, wherein said one or more mesenchymal-like features are selected from the group consisting of expression of vimentin, expression of aSMA, and expression of n-cadherin.
110. The method of any of the preceding paragraphs, wherein after step c) at least a portion of said cancer cells progress to form nodules.
111. The method of any of the preceding paragraph, wherein after step c) at least a portion of said cancer cells transmigrate said membrane.
112. The method of any of the preceding paragraph, wherein said fluidic device is a transwell.
113. The method of any of the preceding paragraph, wherein said fluidic device is a microfluidic device.
114. The method of Paragraph 113, wherein said microfluidic device comprises first and second microchannels separated by said membrane.
115. The method of any of the preceding paragraph, further comprising contacting the healthy cells, the cancer cells, or a combination thereof with at least one agent.
116. The method of Paragraph 115, wherein said agent kills at least a portion of said cancer cells.
117. The method of Paragraph 115, wherein at least a portion of said cancer cells in step c) undergo an epithelial-mesenchymal transition.
118. The method of Paragraph 117, wherein said agent inhibits at least a portion of said cancer cells undergoing said epithelial-mesenchymal transition.
119. The method of Paragraph 110, further comprising contacting the co-culture with an agent that inhibits formation of said nodules.
120. The method of Paragraph 111, further comprising contacting the co-culture with an agent that inhibits said transmigrating of said membrane.

What is claimed is:

1. A method comprising:
providing a first microfluidic device having a body, the body including a first microchannel separated from a second microchannel by an at least partially porous membrane, the membrane having a first side facing the first microchannel and a second side facing the second microchannel;
seeding the first side of the membrane with healthy cells and cancer cells, forming a tissue layer; and
culturing the healthy cells and the cancer cells within the first microfluidic device by flowing fluid through one or more of the first and second microchannels, wherein the density of the cancer cells adhered to the first side of the membrane is in a range such that the culturing of the healthy cells and the cancer cells causes the cancer cells to integrate into the tissue layer formed of healthy cells.

2. The method of claim 1, wherein at least some of said cancer cells and at least some of said healthy cells correspond to the same organ.

3. The method of claim 1, wherein at least some of said cancer cells and at least some of said healthy cells correspond to the different organs.

4. The method of claim 1, wherein said cancer cells are seeded prior to said healthy cells.

5. The method of claim 1, wherein the culturing of the healthy cells and the cancer cells causes the cancer cells to form tight junctions with said healthy cells.

6. The method of claim 1, wherein the healthy cells are differentiated and said cancer cells grow more slowly in the presence of said differentiated healthy cells than in the presence of undifferentiated healthy cells.

7. The method of claim 1, wherein immune cells are included within the tissue layer in the first microchannel and/or a tissue layer in the second microchannel.

8. The method of claim 1, wherein the culturing comprises flowing a culturing medium through the second microchannel while air is present in the first microchannel.

9. The method of claim 1, wherein the culturing comprises flowing a culturing medium through the first and second microchannels.

10. The method of claim 1, wherein the healthy cells seeded in the first channel comprise epithelial cells.

11. The method of claim 1, further comprising seeding second healthy cells in at least a portion of the second microchannel, the second side of the membrane, or a combination thereof.

12. The method of claim 11, wherein said second healthy cells comprise endothelial cells.

13. The method of claim 1, wherein a ratio of the healthy cells to the cancer cells adhered on the first side of the membrane is between about 25:1 and about 500:1.

14. The method of claim 13, wherein the ratio of the healthy cells to the cancer cells adhered on the first side of the membrane is about 100:1.

15. The method of claim 1, wherein a density of the cancer cells adhered to the first side of the membrane is between about 100 to about 10,000 cells/cm2.

16. The method of claim 15, wherein the density of the cancer cells adhered to the first side of the membrane is about 3200 cells/cm2.

17. The method of claim 1, wherein the membrane is coated with at least one attachment molecule that supports adhesion of the healthy cells, the cancer cells, or a combination thereof.

18. The method of claim 1, further comprising applying a fluidic shear force across the membrane within the first microchannel, second channel, or a combination thereof.

19. The method of claim 1, further comprising applying a mechanical force to the healthy cells, cancer cells, or a combination thereof.

20. The method of claim 19, wherein the said applying of a mechanical force comprises applying a mechanical force to the membrane.

21. The method of claim 18, wherein the fluidic shear force controls growth of the cancer cells by inhibiting growth as compared to absence of the fluidic shear force.

22. The method of claim 18, wherein the fluidic shear force, the mechanical force, or combination thereof controls growth of the cancer cells as compared to absence of the said shear force, mechanical force, or the combination thereof.

23. The method of claim 18, wherein the fluidic shear force mimics a shear force of air within a lung during breathing motions.

24. The method of claim 18, wherein the fluidic shear force mimics a shear force of blood flowing through a vessel.

25. The method of claim 19, wherein the mechanical force mimics the expansion and contraction of a lung during breathing motions.

26. The method of claim 19, wherein the mechanical force mimics the motion of at least one portion of the intestine during peristaltic motions.

27. The method of claim 18, further comprising:
applying one or more agents to the healthy cells, the cancer cells, or a combination thereof; and
analyzing the healthy cells, the cancer cells, or a combination thereof to determine effects of the one or more agents.

28. The method of claim 27, wherein the one or more agents are selected from the group consisting of a small molecule, a drug or drug candidate, a chemotherapeutic, a nanoparticle, a compound, a polypeptide, a polynucleotide, a lipid, immunomodulator, and microbes.

29. The method of claim 28, wherein the one or more agents are one or more anti-cancer drugs, and the analyzing is of effects the one or more anti-cancer drugs have on the cancer cells.

30. The method of claim 27, wherein the analyzing comprises detecting the molecular level modulation of drug action.

31. The method of claim 29, wherein the one or more anti-cancer drugs are one or more tyrosine-kinase inhibitors.

32. The method of claim 1, further comprising:
applying the one or more agents to the healthy cells, the cancer cells, or a combination thereof prior to, during, and/or after the application of a fluidic shear force, mechanical force, or a combination thereof; and
analyzing the healthy cells, the cancer cells, or a combination thereof to determine effects of the one or more agents.

33. The method of claim 32, further comprising comparing the effects of the one or more agents applied with and without the application of the fluidic shear force, the mechanical force, or a combination thereof.

34. The method of claim 1, further comprising evaluating the migration of cancer cells between said first and second microchannels.

35. The method of claim 1, wherein the healthy cells are primary cells.

36. The method of claim 35, wherein the primary cells comprise more than one primary cell type.

37. The method of claim 1, wherein the healthy cells are mammalian primary cells.

38. The method of claim 1, wherein the healthy cells are human primary cells.

39. The method of claim 1, wherein the healthy cells are primary epithelial cells.

40. The method of claim 1, wherein the healthy cells are primary endothelial cells.

41. The method of claim 1, wherein the healthy cells are primary stromal cells.

42. The method of claim 1, wherein the healthy cells are primary lung cells.

43. The method of claim 1, wherein the healthy cells are lung alveolar or airway epithelial cells.

44. The method of claim 1, wherein the healthy cells are liver hepatocyte cells.

45. The method of claim 1, wherein the healthy cells are intestinal epithelial cells.

46. The method of claim 1, wherein the healthy cells are sinusoidal endothelial cells.

47. The method of claim 1, wherein the cancer cells are primary cancer cells.

48. The method of claim 47, wherein the primary cancer cells are human primary cancer cells.

49. The method of claim 1, wherein the cancer cells are a cancer cell line.

50. The method of claim 49, wherein the cancer cell line is established from human tissue.

51. The method of claim 1, wherein the cancer cells are lung cancer cells.

52. The method of claim 51, wherein the lung cancer cells are non-small cell lung cancer cells.

53. The method of claim 52, wherein the non-small cell lung cancer cells are non-small cell lung cancer adenocarcinoma cells.

54. The method of claim 1, wherein the cancer cells are metastatic cancer cells.

55. The method of claim 1, wherein the healthy cells and the cancer cells are derived from the same tissue type.

56. The method of claim 1, wherein the healthy cells and the cancer cells are not derived from the same tissue type.

57. The method of claim 1, further comprising contacting the healthy cells, the cancer cells, or a combination thereof with at least one agent.

58. The method of claim 57, further comprising measuring a response of the healthy cells, the cancer cells, or a combination thereof to the at least one agent.

59. The method of claim 58, further comprising extracting the cancer cells from the first microfluidic device prior to measuring the response.

60. The method of claim 57, further comprising measuring products of the cancer cells or healthy cells from an effluent of the first microfluidic device.

61. The method of claim 57, further comprising assessing viability of the cancer cells after the contacting.

62. The method of claim 1, wherein the cancer cells are breast cancer cells, colorectal cancer cells, pancreatic cancer cells, kidney cancer cells, prostate cancer cells, urothelial cancer cells, oesophageal cancer cells, head and neck cancer cells, hepatocellular cancer cells, mesothelioma cells, Kaposi's sarcoma cells, ovarian cancer cells, soft tissue sarcoma cells, glioma, melanoma cells, small-cell and non-small-cell lung cancer cells, endometrial cancer cells, basal cell carcinoma cells, transitional cell carcinoma of the urothelial tract, cervical cancer cells, endometrial cancer cells, gastric cancer cells, bladder cancer cells, uterine sarcoma cells, multiple myeloma cells, soft tissue and bone sarcoma cells, cholangiocarcinoma cells, or a cancer cells disseminated therefrom.

63. The method of claim 1, further comprising imaging the cancer cells within the first microfluidic device.

64. The method of claim 63, further comprising:
modifying the cancer cells to express a fluorescent protein,
wherein the fluorescent protein promotes imaging of the cancer cells.

65. The method of claim 1, further comprising monitoring growth of the cancer cells.

66. The method of claim 1, further providing a second microfluidic device in fluid connection downstream of the first microfluidic device.

67. The method of claim 66, wherein the type of healthy cells comprised in the first microfluidic device and the second microfluidic device are different.

68. The method of claim 66, wherein the flowing medium flows through the first microfluidic device to the second microfluidic device.

69. The method of claim 66, wherein the cancer cells seeded in the first microfluidic device travel to the second microfluidic device.

70. The method of claim 66, wherein the cancer cells seeded in the first microfluidic device integrate into the tissue layer formed of differentiated healthy cells of the second microfluidic device.

71. The method of claim 66, wherein the cancer cells and the healthy cells seeded in the first microfluidic device are derived from the same tissue type.

72. The method of claim 66, wherein the cancer cells and the healthy cells seeded in the first microfluidic device are derived from a different tissue type.

73. The method of claim 66, wherein the cancer cells seeded in the first microfluidic device and the healthy cells seeded in the second microfluidic device are derived from a different tissue type.

74. The method of a claim 66, wherein the healthy cells and the cancer cells seeded in the first microfluidic device are derived from the lung; and the healthy cells seeded in the second microfluidic device are derived from the liver.

75. A method comprising:
a) providing i) cancer cells having one or more mesenchymal-like features, ii) healthy epithelial cells, and a fluidic device comprising a membrane; and
b) co-culturing said cancer cells and said healthy epithelial cells on a first surface of the membrane under conditions such that at least a portion of said cancer cells form tight junctions with said healthy epithelial cells.

76. The method of claim 75, wherein the cancer cells are provided on the membrane at a density range of about 100 to about 10,000 cells/cm².

77. The method of claim 76, wherein the density range controls the growth of the cancer cells compared to outside the density range.

78. The method of claim 76, wherein the cancer cells are provided on the membrane at a density about 3200 cells/cm².

79. The method of claim 75, wherein the cancer cells are provided on the membrane at a ratio of the healthy cells to cancer cells of about 25:1 and about 500:1.

80. The method of claim 79, wherein the ratio controls the growth of the cancer cells compared to outside of the ratio.

81. The method of claim 75, further comprising differentiating said healthy epithelial cells into a differentiated layer, wherein the cancer cells are seeded on said membrane prior to or after differentiating of the healthy cells into the differentiated layer.

82. The method of claim 81, wherein seeding the cancer cells prior to or after differentiating of the healthy cells into the differentiated layer controls the growth of the cancer cells.

83. The method of claim 76, wherein the cancer cells are provided after differentiating of the healthy cells into the differentiated layer.

84. The method of claim 83, wherein seeding with the cancer cells after differentiating of the healthy cells into the differentiated layer controls the growth of the cancer cells to inhibit cancer cell growth.

85. The method of claim 75, further comprising continuing to co-culture until said tumor cells progress to form nodules.

86. The method of claim 75, further comprising contacting the healthy cells, the cancer cells, or a combination thereof with at least one agent.

87. The method of claim 86, wherein said agent kills at least a portion of said cancer cells.

88. The method of claim 85, further comprising contacting the co-culture with an agent that inhibits formation of said nodules.

89. The method of claim 75, wherein said one or more mesenchymal-like features are selected from the group consisting of expression of vimentin, expression of aSMA, and expression of n-cadherin.

90. The method of claim 85, wherein at least a portion of said cancer cells transmigrate said membrane.

91. The method of claim 75, wherein said fluidic device is a transwell.

92. The method of claim 75, wherein said fluidic device is a microfluidic device.

93. The method of claim 75, wherein at least a portion of said cancer cells in step b) undergo a mesenchymal-epithelial transition.

94. A fluidic device comprising:
a membrane; and
a first cell layer formed on a first side of the membrane, the first cell layer comprising first healthy cells and cancer cells, the cancer cells being integrated into the first cell layer and having tight junctions with said healthy cells.

95. The device of claim 94, wherein the first healthy cells are epithelial cells.

96. The device of claim 94, wherein the cancer cells are adhered to the membrane at a cell density of about 100 to about 10,000 cells/cm2.

97. The device of claim 94, wherein the cell density is about 3200 cells/cm2.

98. The device of claim 94, wherein a ratio of the first healthy cells to the cancer cells adhered on the first side of the membrane is between about 25:1 and about 500:1.

99. The device of claim 98, wherein the ratio is about 100:1.

100. The device of claim 94, further comprising a second cell layer formed at least on some portion of the second side of the membrane, the second cell layer comprising second healthy cells.

101. The device of claim 100, wherein said second cell layer comprises endothelial cells.

102. The device of claim 94, further adapted to permit mechanical strain.

103. The device of claim 94, wherein at least some of said cancer cells and at least some of said healthy cells correspond to the same organ.

104. The device of claim 94, wherein at least some of said cancer cells and at least some of said healthy cells correspond to the different organs.

105. The device of claim 94, wherein said fluidic device is a transwell.

106. The device of claim 94, wherein said fluidic device is a microfluidic device

107. The device of claim 106, wherein said microfluidic device comprises a first microchannel and a second microchannel, with the membrane separating the first microchannel from the second microchannel, the membrane having a first side facing the first microchannel and a second side facing the second microchannel.

108. A method comprising:
a) providing i) cancer cells having one or more mesenchymal-like features, ii) healthy epithelial cells, and a fluidic device comprising a membrane;
b) co-culturing said cancer cells and said healthy epithelial cells on a first surface of the membrane under conditions such that at least a portion of said cancer cells form tight junctions with said healthy epithelial cells; and
c) continuing to co-culture until at least a portion of said cancer cells lose said tight junctions with said healthy epithelial cells.

109. The method of claim 108, wherein said one or more mesenchymal-like features are selected from the group consisting of expression of vimentin, expression of aSMA, and expression of n-cadherin.

110. The method of claim 108, wherein after step c) at least a portion of said cancer cells progress to form nodules.

111. The method of claim 108, wherein after step c) at least a portion of said cancer cells transmigrate said membrane.

112. The method of claim 108, wherein said fluidic device is a transwell.

113. The method of claim 108, wherein said fluidic device is a microfluidic device.

114. The method of claim 113, wherein said microfluidic device comprises first and second microchannels separated by said membrane.

115. The method of claim 108, further comprising contacting the healthy cells, the cancer cells, or a combination thereof with at least one agent.

116. The method of claim 115, wherein said agent kills at least a portion of said cancer cells.

117. The method of claim 115, wherein at least a portion of said cancer cells in step c) undergo an epithelial-mesenchymal transition.

118. The method of claim 117, wherein said agent inhibits at least a portion of said cancer cells undergoing said epithelial-mesenchymal transition.

119. The method of claim 110, further comprising contacting the co-culture with an agent that inhibits formation of said nodules.

120. The method of claim 111, further comprising contacting the co-culture with an agent that inhibits said transmigrating of said membrane.
